SEQUENCE LISTING (4 sequences)

SEQ ID NO: 1 - DNA, 720 bp, Artificial Sequence
  Description of Artificial Sequence  Mutant Bcl-2 gene, D34A
atggcgcacgctgggagaacggggtacgacaaccgggagatagtgatgaagtacatccattataagctgtcgcagaggggctacgagtgggatgcgggagctgtgggcgccgcgcccccgggggccgcccccgcaccgggcatcttctcctcccagcccgggcacacgccccatccagccgcatcccgcgacccggtcgccaggacctcgccgctgcagaccccggctgcccccggcgccgccgcggggcctgcgctcagcccggtgccacctgtggtccacctggccctccgccaagccggcgacgacttctcccgccgctaccgcggcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 2 - protein, 239 aa, Artificial Sequence
  Description of Artificial Sequence  Mutant Bcl-2 gene, D34A
MAHAGRTGYDNREIVMKYIHYKLSQRGYEWDAGAVGAAPPGAAPAPGIFSSQPGHTPHPAASRDPVARTSPLQTPAAPGAAAGPALSPVPPVVHLALRQAGDDFSRRYRGDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLSHK

SEQ ID NO: 3 - DNA, 32 bp, Artificial Sequence
  Description of Artificial Sequence  5′ end of Bcl-2 cDNA
gaattcggatcacggtcaccatggcgcacgct

SEQ ID NO: 4 - DNA, 33 bp, Artificial Sequence
  Description of Artificial Sequence  3′ end of Bcl-2 cDNA
ctgagccacaagtgagtcgacctcgaggaattc

(19) United States
(12) Patent Application Publication
(10) Pub. No.: US 2004/0072342 A1
(43) Pub. Date: Apr. 15, 2004

(54) Production and use of microvessels in fibronectin-containing gel

(76) Inventors: Alfred L.M. Bothwell, Guilford, CT (US); Jordan S. Pober, Guilford, CT (US); Jeffrey S. Schechner, Guilford, CT (US); Lian Zheng, New Haven, CT (US)

Correspondence Address: MORGAN LEWIS & BOCKIUS LLP, 1111 PENNSYLVANIA AVENUE NW, WASHINGTON, DC, 20004, US

(21) Appl. No.: 10/297,321

(22) PCT Filed: Jun. 5, 2001
(86) PCT No.: PCT/US01/18034

(51) Int. Cl. C12N 5/08

(52) U.S. Cl. 435/366

(57) ABSTRACT

The present invention relates to the development of new blood vessels. More specifically, this invention relates to compositions and methods for forming cultured endothelial cells into tubes within a three-dimensional gel. This invention also relates to implanting the resultant gels into animals wherein the tubes undergo remodeling into complex microvessels lined by the endothelial cells. The compositions and methods of the present invention have applications in all aspects of tissue and organ transplantation and grafting. The invention finds particular use in the grafting of engineered skin onto recipients with impaired vascularization. In addition, the present invention identifies genes and gene products which are differentially expressed in immature, maturing and mature microvessels.

### FIELD OF THE INVENTION

The present invention relates to the development of new blood vessels. More specifically, this invention relates to compositions and methods for forming cultured endothelial cells into tubes within a three-dimensional gel. This invention also relates to implanting the resultant gels into animals wherein the tubes undergo remodeling into complex microvessels lined by the implanted endothelial cells. The compositions and methods of the present invention have applications in all aspects of tissue and organ transplantation and grafting. The invention finds particular use in the grafting of engineered skin onto recipients with impaired angiogenesis. In addition, the present invention identifies genes and gene products which are differentially expressed in immature, maturing and mature microvessels.

### BACKGROUND OF THE INVENTION

All publications and patent applications herein are incorporated by reference to the same extent as if each individual publication or patent application was specifically and individually indicated to be incorporated by reference.

Angiogenesis is the formation of new blood vessels from established vascular beds. This complex process involves the migration and proliferation of existing vascular endothelial cells (EC), the formation of immature EC tubules, and maturation stages in which mesenchymal cells are recruited and differentiate into the pericytes or smooth muscle cells of the outer vessel layers (Risau (1997) Nature 386, 671-674; Hanahan (1997) Science 277, 48-50; Jain et al. (1997) Nature Medicine 3, 1203-1208).

Many extracellular matrix (ECM) components and soluble factors that promote, mediate or inhibit angiogenesis have been identified. Through integrin and growth factor receptors on endothelial cells, these regulators activate intracellular signaling cascades that suppress or cause apoptosis of the proliferating vascular cells. Several pathways have been identified which support angiogenesis by triggering the production of the anti-apoptotic Bcl-2 protein within the EC. The angiogenic effects of ECM components such as collagen and fibronectin also include anti-apoptotic effects exerted through these pathways.

Angiogenesis plays a significant role in wound healing, tumor growth, cardiovascular disease, and tissue transplantation. Patients with thermal burns or venous leg ulcers, or acute or chronic wounds, and populations such as diabetics and the elderly suffer tissue damage due to ischemia for which induced revascularization might offer some relief. The clinical use of engineered skin for treatment of burns has met with moderate success, but is limited by the lack of vascularization and consequent sloughing of the synthetic dermal and epidermal layers. Stimulation of angiogenesis by the introduction of endothelial cells into these compromised tissues shows promise but current efforts are hampered by poor endothelial cell survival, and a lack of maturation of the primitive vascular tubes they form. Consequently, significant effort has been directed at developing models in which to study and manipulate these processes.

Advances in understanding the mechanism of early vascular remodeling have come from the ability to successfully suspend the endothelial cells (EC) in three-dimensional (3-D) culture, where they form tubular structures that resemble immature capillaries (Springhorn et al. (1995) In Vitro Cell. Dev. Biol. Anim. 31, 473-481; Madri et al. (1992) Kidney Int. 41, 560-565) and, unlike conventional two-dimensional culture, exhibit phenotypes comparable to endothelial cells in vivo (Madri et al. (1992) Kidney Int. 41, 560-565). Such models have been applied to assessing the effects of soluble factors such as vascular endothelial growth factor, leptin, or angiopoietin-1 on vascular remodeling (Papapetropoulos et al. (1997) J. Clin. Invest. 100, 3131-3139; Sierra-Honigmann et al. (1998) Science 281, 1683-1686, and Papapetropoulos et al. (1999) Lab. Invest. 79, 213-223). These 3-D culture systems have been particularly useful in analyzing the interactions between matrix molecules and EC (Merwin et al. (1990) J. Cell Physiol. 142, 117-128; Sankar et al. (1996) J. Clin. Invest. 97, 1436-1446).

Reports of models useful for discerning the mechanisms of incorporation of mesenchymal cells into mature vessels have been limited. Co-culture of canine brain EC with astrocytes suspended in a collagen matrix has resulted in the formation of complex vessel-like structures composed of both cell types (Ment et al. (1997) In Vitro Cell. Dev. Biol. Anim. 33, 684-691). Other studies on the recruitment and incorporation of pericytes and smooth muscle cells, although useful in identifying receptor-ligand pairs that may be important in these processes, have either been two dimensional (Hirschi et al. (1999) Circ. Res. 84, 298-305), or have addressed remodeling associated with vasculogenesis during embryonic development, rather than angiogenesis in adults (Thurston et al. (1999) Science 286, 2511-2514; Sato et al. (1995) Nature 376, 70-74; and Shalaby et al. (1995) Nature 376, 62-66).

We have invented methods for the construction of synthetic human vascular beds that allows genetic manipulation of endothelial cells to improve perfusion in vivo. Our invention provides models for further analyses of the mechanisms of complex vascular organization, as well as for developing stable engineered vascular beds that can be utilized to perfuse transplanted human tissue. Major limitations in the construction of human synthetic microvessels include the apoptotic response of cultured human endothelial cells before or after the formation of immature tubes (Ilan et al. (1998) J. Cell. Sci. 111, 3621-3631), and the lack of progress to more mature tubule structure and function involving the recruitment and differentiation of nearby mesenchymal cells.

Interaction with matrix components through the integrins α₅β₁ or αᵥβ₃ has been shown to inhibit EC apoptosis in culture (Fukai et al. (1998) Exp. Cell Res. 242, 92-99) and in vivo (Brooks et al. (1994) Cell 79, 1157-1164). Increased expression of the survival gene Bcl-2 also appears to play a role in preventing the involution of synthetic capillary networks (Pollman et al. (1999) J. Cell Physiol. 178, 359-370). Overexpression of Bcl-2 by retroviral transduction not only resulted in prolonged survival of human dermal microvascular EC, but also allowed incorporation of human EC into newly formed mouse capillaries in vivo (Nor et al. (1999) Am. J. Pathol. 154, 375-384.).

This invention optimizes the methodology for induction of tube formation by cultured EC within 3-D gels, and for successful inosculation of these preformed networks of cultured cells with the circulatory system of a host. The invention includes the construction of a simple extracellular matrix composed of type I collagen plus human plasma fibronectin. The survival of, and the tube formation by, cultured human umbilical vein EC (HUVEC) in such constructs was improved by transduction with a modified (caspase-resistant) form of Bcl-2 protein (Cheng et al. (1997) Science 278, 1966-1968). Such cultured HUVEC are consistently incorporated into the mouse circulatory system. Furthermore, overexpression of caspase-resistant Bcl-2 in these cells results in the formation perfused vascular structures invested by mouse pericyte/smooth muscle cells, that remodel into mature vessels.

### SUMMARY OF THE INVENTION

This invention provides synthetic compositions comprising collagen, fibronectin, and at least one cell. More specifically, this invention provides such synthetic compositions wherein the cell is an endothelial cell that comprises a nucleic acid encoding one or more anti-apoptotic proteins. Even more specifically the nucleic acid encodes a cleavage-resistant Bcl-2 polypeptide. Most preferably, this invention provides such synthetic compositions wherein the nucleic acid encodes a caspase-resistant Bcl-2 polypeptide designated D34A. Other anti-apoptotic proteins that may be included in the compositions of the invention in place of or in addition to the D34A form of the Bcl-2 protein, include but are not limited to the D31A form of Bcl-2, proteins from the family of “Bcl-2-related” anti-apoptotic proteins, A20, and IAP-related proteins such as survivin.

The endothelial cells utilized in the compositions of the invention can be derived from many sources, including, but not limited to, human umbilical veins, human dermal microvascular beds, adipose tissues and porcine tissues.

This invention further provides such compositions wherein the composition is a solution capable of gelation, or a semi-solid solution capable of gelation, or a gel. A preferred composition is one wherein the collagen is Type I collagen. Another preferred composition is one in which the fibronectin is human plasma fibronectin. The invention also includes compositions in which digested fibronectin, fragments of fibronectin, and/or RGD-motif containing peptides are included in place of or in addition to intact fibronectin.

This invention also provides constructs prepared by methods which include preparing a solution comprising collagen and fibronectin; suspending endothelial cells in the solution, wherein the suspended endothelial cells comprise a nucleic acid encoding a caspase-resistant Bcl-2 polypeptide and/or other anti-apoptotic protein; and polymerizing the collagen within the solution to form a three-dimensional gel.

This invention also provides constructs prepared by methods which include preparing a solution comprising collagen and fibronectin; suspending endothelial cells in this solution, wherein the suspended endothelial cells comprise a nucleic acid encoding a caspase-resistant Bcl-2 polypeptide; adjusting the solution to between about pH 7.0 and about pH 8.0; and warming the solution to between about 25° C. and about 40° C. to form a three-dimensional gel. In a more preferred embodiment, the pH is adjusted to between about pH 7.2 and about pH 8.0. In an even more preferred embodiment, the pH is adjusted to between about pH 7.4 and about pH 7.6. In another preferred embodiment, the solution is warmed to between about 30° C. and about 38° C. In another even more preferred embodiment, the solution is warmed to between about 36° C. and about 38° C. The invention also includes all combinations of these pH and temperature options.

The present invention also provides methods for vascularizing acellular dermis by seeding the acellular dermis with endothelial cells. Specifically, the endothelial cells may include cells that comprise nucleic acids encoding a caspase-resistant Bcl-2 polypeptide and/or other bcl-related genes and anti-apoptotic genes. This invention also includes those methods that further comprise the additional step of implanting seeded acellular dermis into an animal. More specifically, the invention provides such methods wherein the animal is an immunodeficient animal. Even more specifically, the invention provides such methods wherein the immunodeficient animal is a SCID or SCID/beige mouse.

This invention further provides methods for vascularizing acellular dermis with human endothelial cell lined blood vessels using the constructs of the present invention. Specifically, HUVEC are used to seed acellular dermis which when implanted into an animal, especially an immunodeficient animal, become perfused with blood. The endothelial cells used in such methods preferably include cells that comprise nucleic acids encoding a caspase-resistant Bcl-2 polypeptide and/or other bcl-related genes and anti-apoptotic genes.

The present invention additionally provides methods of using retroviral transduction to genetically manipulate the endothelial cells incorporated into the skin substitutes of the present invention, wherein the transduction is used to add functionality to the skin substitutes. For example, overexpression of the survival gene Bcl-2 in the HUVEC results in increased graft perfusion. Other uses of genetic manipulation of the EC incorporated in skin equivalents according to the present invention include, but are not limited to, resistance to graft rejection, drug delivery, and improved angiogenesis. Besides the clinical applications, the skin equivalents of the present invention may also be used as experimental models to study interactions between different skin cells, and the interactions between skin cells and the immune system. The capacity for genetic manipulation of the cells incorporated in these constructs, and even the selective inclusion of different cells, offer significant advantages over presently available models using whole skin.

This invention further provides methods of animal implantation which include implanting into an animal the constructs produced by such methods. More specifically, the invention provides such methods wherein the animal is an immunodeficient animal. Even more specifically, the invention provides such methods wherein the immunodeficient animal is a SCID or SCID/beige mouse.

This invention also provides methods for forming endothelial cells into tubes within a matrix which include the steps of preparing a solution comprising collagen and fibronectin; suspending endothelial cells in the solution, wherein the suspended endothelial cells comprise a nucleic acid encoding a caspase-resistant Bcl-2 polypeptide; polymerizing the collagen within the solution to form a matrix; and allowing the endothelial cells to form tubes within the matrix.

The present invention also provides methods of producing endothelial cell tubules in vivo which include the steps of preparing a solution comprising collagen and fibronectin; suspending endothelial cells in the solution, wherein the suspended endothelial cells comprise a nucleic acid encoding a caspase-resistant Bcl-2 polypeptide; warming the suspension so that the collagen gels to produce a three-dimensional gel; polymerizing the collagen within the solution to form a three-dimensional gel; and implanting the three-dimensional gel produced into an animal. More specifically, the invention provides such methods wherein the animal is an immunodeficient animal. Even more specifically, the present invention provides such methods wherein the immunodeficient animal is a SCID or SCID/beige mouse. In one preferred embodiment, the invention provides such methods wherein the endothelial cell tubules have one or more characteristics of mature microvessels. In particular, the invention provides such methods wherein the cells surrounding the human endothelial cells are derived from the animal into which the three-dimensional gel is subsequently implanted. In another preferred embodiment, the invention provides such methods wherein the endothelial cell tubules are perfused by blood. The present invention also provides endothelial cell tubules produced by such methods. More specifically, the invention provides such endothelial cell tubules wherein the endothelial cell tubules are perfused by blood.

The present invention also provides methods of promoting vascularization in a tissue or an organ, including the steps of preparing a solution comprising collagen and fibronectin; suspending endothelial cells in the solution, wherein the suspended endothelial cells comprise a nucleic acid encoding a caspase-resistant Bcl-2 polypeptide; polymerizing the collagen within the solution to form a three-dimensional gel; allowing the endothelial cells in the three-dimensional gel to form tubules; and contacting the tubule-containing three-dimensional gel with a tissue. This invention further provides such methods wherein the tissue or organ is used for engraftment. More specifically, the present invention provides such methods wherein the tissue is synthetic skin.

The present invention also provides methods of promoting vascularization/revascularization in an animal, which includes the steps of preparing a solution comprising collagen and fibronectin; suspending endothelial cells in the solution, wherein the suspended endothelial cells comprise a nucleic acid encoding a caspase-resistant Bcl-2 polypeptide; and directly injecting the solution into an animal of choice, including humans.

The present invention also provides each of the above methods wherein at least some of the cells of the recipient cell cultures, tissues, organs or whole animals have been transduced with Bcl-2, including caspase-resistant Bcl-2, prior to the implantation or direct injection of the constructs of the present invention into such cell cultures, tissues, organs or whole animals. More specifically, the present invention provides each of the above methods wherein at least some of the cells of a cell culture, tissue or organ which come in contact with the implanted or injected constructs of the present invention have been transduced with Bcl-2, including caspase-resistant Bcl-2, prior to the implantation or direct injection of the constructs of the present invention into such cell cultures, tissues or organs.

The present invention provides methods for identifying genes and/or gene products involved in the process of vascularization which include obtaining a first construct comprising endothelial cells wherein the construct promotes vascularization; obtaining a second construct comprising endothelial cells wherein the construct does not promote vascularization; and identifying gene products detectable in one but not both constructs.

The present invention also provides methods for identifying genes and/or gene products involved in the process of vascular remodeling which include obtaining a first construct comprising endothelial cells wherein the construct promotes vascular remodeling; obtaining a second construct comprising endothelial cells wherein the construct does not promote vascular remodeling; and identifying gene products detectable in one but not both constructs.

The present invention also provides methods of identifying agents that modulate vascularization or vascular remodeling which include the steps of exposing a construct to an agent, wherein the construct comprises endothelial cells and promotes vascularization or vascular remodeling; and determining whether the agent modulates vascularization or vascular remodeling. The identification methods described above can be conducted in vivo or in vitro.

### BRIEF DESCRIPTION OF THE DRAWINGS

FIG. 1. Expression of MHC class I on EGFP or Bcl-2 transduced HUVEC are not altered. Uninfected HUVEC control, EGFP or Bcl-2 transduced HUVEC were incubated with mAb to MHC class I (dashed line) or control mAb (solid line) and stained with a PE-conjugated Donkey anti-mouse secondary Ab. Fluorescence was quantitated by a FACScan flow cytometer.

FIG. 2. Expression of EGFP and Bcl-2 in transduced HUVEC. EGFP transduced HUVEC were analyzed directly by flow cytometry using FL1 for GFP expression (A). For detection of Bcl-2 expression, both EGFP (B-a) and Bcl-2 (B-b) transduced HUVEC were incubated with mAb to Bcl-2 (dashed line) or control mAb (solid line), and stained with a PE-conjugated Donkey anti-mouse secondary Ab. Fluorescence was quantitated by a FACScan flow cytometer. These data are representative of data from three independent transductions.

FIG. 3. Response of transduced HUVEC to withdrawal of growth factor and serum. HUVEC monolayers were cultured in the absence of growth factor and serum for four days. Cell killing was then measured. Each data point represents the mean of triplicate samples ±SE.

FIG. 4. Bcl-2 protects HUVEC from apoptotic death induced by growth factor and serum deprivation. HUVEC-EGFP (A, C) and HUVEC-Bcl-2 (B, D) were cultured in M199 medium with (A, B) or without growth factor and serum (C, D). After a 24 hour incubation, HUVEC were stained with DAPI and photographed through a fluorescence microscope.

FIG. 5. Bcl-2 protects HUVEC from apoptosis induced by staurosporine. HUVEC monolayers were treated with staurosporine for 24 hours. Cell killing was then measured. Each data point represents the mean of triplicate samples ±SE. The experiment shown is representative of three similar experiments.

FIG. 6. Bcl-2 protects HUVEC from apoptosis induced by ceramide ±TNF-α. HUVEC monolayers were treated with C-6-ceramide in the absence (A) or presence of TNF-α (B) for 24 hours. Cell killing was then measured. Each data point represents the mean of triplicate samples ±SE. The experiment shown is representative of three similar experiments.

FIG. 7. Overexpression of Bcl-2 protects HUVEC from alloreactive CTL. CTL generated by allogeneic BLCL stimulators were used as effectors against transduced HUVEC targets derived from the same donor as BLCL. Each data point represents the mean of triplicate samples ±SE. The experiment shown is representative of three similar experiments.

FIG. 8. Redirected cytolysis is inhibited by Bcl-2. Redirected cytolytic activity were assayed in the presence of 5 μg/ml of PHA (phytohaemagluttinin) using transduced HUVEC targets derived from donors different than that of BLCL stimulators. No cytolytic activity of the third party donors was observed in the absence of PHA. Each data point represents the mean of triplicate samples ±SE.

FIG. 9. The behavior of untransduced HUVEC in 3-D gel culture, and in synthetic vascular beds in vivo. A. Phase contrast microscopy of untransduced HUVEC in 3-D gel culture, 24 hours after suspension in a collagen fibronectin gel (400×)*. (B) EM of these constructs showing the HUVEC form aggregates with a lumen like (L=lumen) configuration cleared of matrix proteins (10,000×). (C) EM of another field of the same construct, containing a cell undergoing apoptosis as demonstrated by condensation of the nucleus (10,000×). (D) Untransduced HUVEC construct harvested 31 days after implantation into a SCID-beige mouse (Hematoxylin and eosin (H+E) stain, 400×). (E) UEA-1 reactivity is seen on the cells lining the vascular spaces (200×). (F) A mock construct in which HUVEC were not included in the gel (H+E stain, 200×). *All magnifications are reported as original magnification prior to photographic enlargement.

FIG. 10. The behavior of retrovirally transduced HUVEC in vitro. H+E staining of (A) EGFP- and (B) Bcl-2-transduced HUVEC, 24 hours after suspension in 3-D gel culture (200×). (C) Intrinsic fluorescence of an EGFP-transduced (and inset absence of signal from Bcl-2-transduced) HUVEC construct (100×). (D) Anti Bcl-2 antibody staining of the Bcl-2-transduced (brown, and inset EGFP-transduced) construct at this same time point (200×). (E, F) Phase contrast microscopy of the EGFP-(E) and Bcl-2-(F) transduced HUVEC maintained in 3-D gel culture for 36 hours (400×). After seven days in 3-D gel culture there are no detectable viable EGFP-transduced cells (G), while those transduced with Bcl-2 are still organized into cords (H) (400×).

FIG. 11. The behavior of retrovirally transduced HUVEC in vivo. Histology of (A) Bcl-2- and (B) EGFP-transduced HUVEC constructs harvested 31 days after implantation into a SCID-beige mouse (1000×). UEA-1 staining of the constructs in panels (A) and (B), (C & D, respectively) (200×). (E) Anti Bcl-2 staining (brown) of a Bcl-2-transduced construct 31 days after implantation into a SCID-beige mouse (100×). (F) Fluorescence of the EGFP-transduced HUVEC constructs in vivo (400×).

FIG. 12. Analysis of complex vascular structures. Double immunostaining of frozen sections for UEA-1 (red) and smooth muscle α-actin (blue) in (A) Bcl-2- and (B) EGFP-transduced constructs harvested from mice 31 days after implantation. (200×). C. UEA-1 staining (brown) of a larger vessel from a Bcl-2 transduced construct 31 days after implantation (400×). (D) Smooth muscle α-actin staining (brown) of this same construct (400×). (E) Histology of a Bcl-2-transduced construct harvested 60 days after implantation. (F) UEA-1 staining (brown) of this same construct, [symbol] =arterial like structure, [symbol] =venous like structure, *=capillary like structure (400×).

FIG. 13. EM of constructs 31 days after implantation into SCID/beige mice. (A) Untransduced HUVEC have formed perfused vessel like structures which have inosculated with the mouse circulation as demonstrated by the presence of erythrocytes within the lumen. The vessel has a single endothelial layer surrounded by matrix. (B) The Bcl-2 transduced HUVEC have formed more complex vessels which are now comprised of the EC layer surrounded by a second layer representing a pericyte/smooth muscle cell. (C) This vessel formed from Bcl-2 transduced HUVEC shows an even more complex structure with an endothelial layer surrounded by several layers of investing cells, mimicking the anatomy of a post capillary venule. (EC=Endothelial cells, RBC=erythrocytes, *=investing cell, 10,000×).

FIG. 14. Bcl-2 and EGFP expression in transduced PAEC at one and two months post-implantation of collagen/fibronectin gels into SCID/beige mice. (A) H+E staining of EGFP at one month. (B) Staining with anti-EGFP Ab at one month. (C) H+E staining of Bcl-2 at one month. (D) Staining with anti-Bcl-2 Ab at one month. (E) Staining with anti-smooth muscle α-actin at one month. (F) H+E staining of Bcl-2 at two months. (G) Staining with anit-Bcl-2 at two months. (H) Staining with anti-smooth muscle α-actin at two months.

FIG. 15. (A) Human Endothelial specific UEA-1 stain of human acellular dermis 72 hours after seeding with cultured human umbilical vein endothelial cells (HUVEC), but prior to implantation in a mouse. The positive reactivity is consistent with repopulation of the vascular channels by the cultured cells in vitro.

FIG. 16. UEA-1 stain of acellular dermis seeded with HUVEC one month after implantation into a scid/beige mouse. The positive reactivity (red) indicates that the vascular structures are lined by human endothelial cells.

FIG. 17. H+E stain of acellular dermis seeded with HUVEC one month after subcutaneous implantation into a scid/beige mouse. This figure demonstrates that the human endothelial lined microvessels shown in FIG. 16 contain mouse erythrocytes, indicating that they are perfused by mouse blood.

FIG. 18. H+E stain of acellular dermis that has not been seeded with HUVEC harvested one month after implantation into a SCID/beige mouse. Without HUVEC, there is no significant vascularization of the acellular dermis in vivo, indicating that successful perfusion is dependent on the seeding with human endothelial cells prior to implantation.

FIG. 19. Anti-Bcl-2 stain of acellular dermis seeded with Bcl-2 transduced HUVEC, 1 month after subcutaneous implantation in a scid/beige mouse. The positive reactivity (red) of all the vascular structures demonstrates the persistent expression of the transgene in vivo.

FIG. 20. Human endothelial and keratinocyte specific UEA-1 stain (A) and human endothelial and basement membrane specific type IV collagen stain (B, both red) of acellular dermis seeded with human keratinocytes and endothelial cells, thirty days after subcutaneous implantation into SCID/beige mice. Note the presence of a stratified and keratinized epidermis (E) with underlying dermal vessels that are perfused by murine blood and lined with human endothelium (*).

### DETAILED DESCRIPTION OF THE PREFERRED EMBODIMENTS

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

“Acellular dermis” as used herein, refers to a layer of skin composed of connective tissue located below the epidermis, wherein all of the cellular components have been removed.

“Agent” as used herein, refers to anything which is applied to a cell of interest, including, but not limited to, peptides, polypeptides, nucleic acids, any other organic compound or any inorganic compound.

“Angiogenesis” as used herein, refers to the formation of new blood vessels from established vascular beds.

“Construct” as used herein, generally refers to a matrix and whatever originates, develops or is contained in the matrix. More specifically and as used herein, “construct” refers to a matrix in which cells have been seeded. The matrix of the construct is typically a three-dimensional collagen-based gel which may contain fibronectin and/or one or more other components such as cells, buffers, salts, extra cellular proteins, growth factors, etc.

“Gel” as used herein, refers to the solid or semisolid phase of a colloidal solution.

“Gene product” as used herein, refers to any of the types of RNA (transcription products) or any of the proteins or protein subunits (translation products) synthesized biochemically on the basis of the information encoded by nucleic acids.

“Matrix” as used herein, refers to the surrounding substance within which something else originates, develops or is contained.

“Organ” as used herein, refers to a differentiated part of an organism which with a specific function. Examples include, but are not limited to, parts which have specific functions such as respiration, secretion or digestion.

“Peptide” as used herein, refers to any compound containing two or more amino-acid residues joined by amide bond(s). Unless stated otherwise herein for a specific context, the term peptide can be used interchangeably with polypeptide or protein.

“Polypeptide” as used herein, refers to a polymer made up of less than about 50 amino acids. Unless stated otherwise herein for a specific context, there is no minimum number of amino acids which must be present in order for a polymer to be classified a polypeptide. Also, unless stated otherwise herein for a specific context, the term polypeptide can be used interchangeably with peptide or protein.

“Protein” as used herein, refers to a molecule composed of one ore more polypeptide chains. Unless stated otherwise herein for a specific context, the term protein can be used interchangeably with peptide or polypeptide.

“Three-dimensional cell culture” or “3-D cell culture” as used herein, refers to cell cultures wherein cell expansion can occur in any direction as long as the cells are not at the edge of the culture.

“Tissue cell culture” as used herein refers to an aggregation of cells and intercellular matter performing one or more functions in an organism. Examples of tissues include, but are not limited to, epithelium, connective tissues (e.g., bone, blood, cartilage), muscle tissue and nerve tissue.

“Two-dimensional cell culture” or “2-D cell culture” as used herein, refers to conventional monolayer cell culture. Generally, every cell in a 2-D culture directly contacts the substratum on the plate and the cultures, therefore, only expand horizontally as they proliferate.

“Vascularization” as used herein, refers to the formation of new blood vessels.

“Vascular remodeling” as used herein, refers to the maturation of endothelial cell tubules into complex endothelium-lined microvessels invested with mesenchymal cells such as pericytes and smooth muscle cells. The presence of the smooth muscle cells can be determined by measuring smooth muscle α-actin expression.

Cell Isolation and Conventional (2-D) Cell Culture

General techniques of mammalian cell isolation and culture are well established, see, for example, Freshney (2000) Culture of animal cells: a manual of basic technique, John Wiley; Freshney (1992) Culture of epithelial cells, John Wiley; Mather et al. (1998) Introduction to cell and tissue culture: theory and technique, Plenum Publishers; Harrison et al. (1997) General techniques of cell culture, Cambridge University Press.

Long-term mammalian cell culture has been difficult to achieve. Many types of specialized cells plated on standard tissue culture plastic dishes dedifferentiate, lose function, and fail to proliferate. The importance of the extracellular matrix and extracellular matrix molecules in maintaining cell function and allowing cell growth have been described by, for example, Jauregui et al. (1986) In Vitro Cell. Dev. Biol. 22, 13-22; Kleinman et al. (1987) Anal. Biochem. 166, 1-13; Mooney et al. (1992) J. Cell. Physiol. 151, 497-505.

While the adult endothelium in vivo is remarkably quiescent, ECs can be induced to proliferate, e.g., following traumatic injury, inflammation, and tumor formation or in response to physiologic cues during hair growth and ovarian cycling. This property has allowed the in vitro cultivation and expansion of ECs. Many endothelial cell lines may now be obtained commercially, including human saphenous vein ECs (e.g., Vascular Endothelial Cell (VEC) Laboratories), human aortic ECs, human coronary arterial ECs and human dermal microvascular ECs (e.g., Clonetics). However, immortal endothelial cell lines generated by viral or spontaneous transformation invariably fail to exhibit characteristic markers and physiologic responses and eventually lose important differentiated EC functions. Such markers include the cell surface expression of E-selectin and CD31 (PECAM-1) and the formation of tubule-like structures in response to matricellular signals, in three-dimensional culture. As a result, cultured ECs are typically primary cultures. Vascular and microvascular endothelial cells from humans and animals have been harvested and studied from a variety of tissues, and heterogeneity of microvascular endothelial cell antigen expression and cytokine responsiveness has been noted in situ and in cell culture (Petzelbauer et al. (1993) J. Immunol. 151, 5062-5072).

In 1973 Jaffe et al., successfully cultured endothelial cells from human umbilical veins (HUV) and these cells, known as HUVECs, have been characterized functionally (Jaffe et al. (1973) J. Clin. Invest. 55, 2757-2764; Lewis (1972) Am. J. Anat. 30, 39-59; Jaffe et al. (1973) J. Clin. Invest. 52,2745-2756). Current methods of HUVEC isolation and conventional monolayer (two-dimensional) culture may use collagenase to disrupt the source tissue, gelatinized plates and serum and EC supplement to maintain the cells (Gimbrone (1976) Prog. Hemostasis Thromb. 3, 1-6; Zheng et al. (2000) J. Immunol. 164, 4665-4671).

Human microvascular endothelial cells, which differ from large vessel endothelial cells, and which also vary depending on the type of tissue from which they are derived, have also been isolated from lung tissue and characterized (Lou et al. (1998) In Vitro Cell. Dev. Biol. Anim. 34, 529-536; Chen et al. (1995) Microvasc. Res. 50, 119-128). In adipose tissue, Hewett et al. (1993) In Vitro Cell. Dev. Biol. Anim. 29A, 325-331 have developed a method for isolating and characterizing microvessel endothelial cells from human mammary glands, and Springhorn et al. (1995) In Vitro Cell. Dev. Biol. Anim. 31, 473-481 have examined human capillary endothelial cells from the abdominal wall. The isolation of vascular endothelial cells from non-human lung tissue has also been described, including manual and automated methods for isolating vascular endothelial cell from the omentum in dogs (Pasic et al. (1996) Eur. J. Cardiothoracic Surg. 10, 372-379).

Porcine aortic endothelial cells (PAEC) may be obtained commercially at passage one (e.g., Cell Systems) and cultured as monolayers in, for example, DMEM containing 10% FBS, penicillin 100 U/ml and streptomycin 100 μg/ml (JRH Biosciences) also referred to as D10 medium (U.S. Pat. No. 5,891,645). Alternative methods of PAEC monolayer culture are also effective, such as the method described, for example, in U.S. Pat. No. 5,977,076 or in Maher et al. (1996) J. Immunol. 157, 3838-3844.

Three-Dimensional (3-D) Cell Culture

Like most human somatic cells ECs undergo replicative senescence in vitro after a finite number of divisions, which varies depending on the tissue of origin and culture condition. Efforts to prolong EC survival have included the addition of exogenous growth factors, overexpression of telomerase, and the provision of supportive matrix components. (Yang et al. (1999) J. Biol. Chem. 274, 26141-26148; Yang et al. (2000) J. Invest. Derm. 114, 765-768; Bicknell (1996) Endothelial cell culture, Cambridge University Press).

When harvested microvascular endothelial cells are plated on two-dimensional (2-D) substrata, as in conventional cell culture, they proliferate until they form a tightly apposed confluent monolayer of quiescent cells that display a typical “cobblestone” morphology. In this environment they lose the arc of curvature normally seen in vivo and become flattened cells exhibiting altered phenotypes and associations with the substratum that are not similar to the associations with the specific extracellular matrix components and pericytes of their 3-D environment in vivo (Madri et al. (1991) J. Cell. Biochem. 45, 1-8). A more differentiated phenotype returns if collagen is provided as a component of a gelatin-based substratum. In tumor-conditioned medium, ECs on a collagen-coated culture dish can spontaneously develop internal vacuoles that join up, eventually giving rise to a network of capillary tubes (Folkman et al. (1980) Nature 288, 551-556). Surface-attached tubular elements were formed on a fibronectin-coated culture dish in the presence of EC growth factor (ECGF) with one HUVEC cell forming the circumference of the lumen. These conditions also reduced the serum requirement for growth and permitted serial propagation of the HUVEC, which otherwise do not proliferate beyond two or three passages (Maciag et al. (1982) J. Cell Biol. 94, 511-520). Nonetheless, these networks are limited to the two-dimensional surface of the culture dish and do not approximate in vivo conditions and phenotypes as closely as can be achieved with a 3-D culture system.

Several 3-D culture systems have been devised that allow the formation of three-dimensional cellular networks (also known as constructs) that resemble immature capillary beds. When microvascular endothelial cells are dispersed and cultured in a 3-D type I collagen gel, exposure to growth factors prompts a distinct and dramatic morphological change. Individual ECs display an elongated “sprouting” morphology and an arc of curvature, and undergo formation of multicellular tube-like structures having junctional complexes and luminal specializations. When the EC are isolated from a fenestrated vascular bed, they form fenestrated tube-like structures if given an appropriate matrix. (Springhorn et al. (1995) In Vitro Cell. Dev. Biol. Anim. 31, 473-481; Madri et al. (1992) Kidney Int. 41, 560-565; Merwin et al. (1990) J. Cell Physiol. 142, 117-128; Madri et al. (1986) J. Histochem. Cytochem. 34, 85-91). Methods of 3-D culture in a type I collagen may be further refined, for example by using isolation methods that enhance the purity of tubule-forming EC through active selection of EC markers (Springhorn et al. (1995) In Vitro Cell. Dev. Biol. Anim. 31, 473-481), by the inclusion of growth or anti-apoptotic factors, or by other anti-senescence approaches that address the problem of poor EC survival in vitro (for example, see Papapetropoulos et al. (1997) Clin. Invest. 100, 3131-3139; Sierra-Honigmann et al. (1998) Science 281, 1683-1686; Papapetropoulos et al. (1999) Lab. Invest. 79, 213-223; Merwin et al. (1990) J. Cell Physiol. 142, 117-128; Sankar et al. (1996) J. Clin. Invest. 97, 1436-1446; Nor et al. (1999) Am. J. Pathol. 154, 375-384; Yang et al. (1999) J. Biol. Chem. 274,26141-26148).

Extracellular Matrix (ECM) Proteins

The extracellular matrix (ECM) is a layer consisting mainly of proteins (especially collagen) and glycosaminoglycans (mostly as proteoglycans) that form a sheet underlying cells such as endothelial and epithelial cells. The constituent substances are secreted by cells in the vicinity, especially fibroblasts. Examples of ECM proteins include, but are not limited to, fibronectin, collagen, laminin, vitronectin, thrombospondin, von Willebrand factor, fibrinogen, tenascin, osteopontin and the like, and cell-surface binding fragments and analogs thereof Collagen and fibronectin are the two most important of the ECM proteins for the purposes of this invention.

Collagen

Collagen is a fibrous protein that form fibrils having a very high tensile strength and that has been found in most multicellular organisms. Collagen serves to hold cells and tissues together and to direct the development of mature tissue. Collagen is the major fibrous protein in skin, cartilage, bone, tendon, blood vessels and teeth.

There are many types of collagen which differ from each other to meet the requirements of various tissues. Some examples of types of collagen are as follows: type one [a1(I)₂]a.2 which is found in skin, tendon, bone and cornea; type two [a1(II)]₃ which is found in cartilage intervertebral disc, and the vitreous body; type three [a1(III)]₃ which can be found in skin and the cardiovascular system; type four [a1(IV)]₂a2(IV) which can be found in basement membrane; type five [a1(V)]₂a2(V) and a1(V)a2(V)a3(V) which is found in the placenta and cornea. Examples of newly identified forms of collagen include: type seven (VII) which is found in anchoring fibrils beneath many epithelia; and types nine (IX), ten (X) and eleven (XI), which are minor constituents of cartilage (U.S. Pat. No. 5,064,941).

In one embodiment, collagen can be isolated from rat tail tendons, rabbit and bovine tendons, corneas and placentas. In a related aspect, conditions whereby collagen can be extracted from are: (1) low ionic strength and neutral buffer; (2) weak acid solution; and (3) partial pepsin digestion followed by extraction in acid solution. For example, the collagen can be derived by acid extraction followed by salt precipitation of rat tail collagen from acid solution. By avoiding the use of pepsin, collagen retains intact telo-peptides and the ability to form lysine-derived covalent crosslinks (U.S. Pat. No. 5,756,350).

Preferably, the collagen is type I collagen from 6-12 week rat tail tendon. Rat tendons are cleanly dissected from the tail that is iced, skinned and briefly washed with 1.0 mM benzamidine hydrochloride and 5.0 mM ethylenediaminetetraacetic acid (EDTA) in 0.1M NaCl to inhibit proteolysis as described in Ghosh (1988) Connect. Tiss. Res. 17, 33-41. The tendons are frozen on dry ice and then powdered in liquid nitrogen in a Wiley mill. The tissues or tissue powders are soaked overnight at 4° C. in 1.0 M ethylenediamine hydrochloride (pH 8) or hydrochloride salts of the other solvents, and then sheared through a Dounce homogenizer several times to obtain a uniform slurry. Optionally, mercaptoethanol is added at this time and tissues are stirred for 24 hours before a second homogenization with the Dounce homogenizer and centrifugation at 36,000×g for forty minutes or 83,000×g for one hour in a Beckman SW 28 Ti rotor. The clear supernatant is decanted, the viscous residue is redispersed in fresh solvent, and the extraction process is repeated two or three times (U.S. Pat No. 5,064,941).

In another embodiment, rat tail tendon is prepared by a modification of a procedure described by Elsdale et al. (1972) J. Cell Biol. 54, 626-637. Briefly, four tendons are dissected from each rat tail and are left stirring in 200 ml of 3% acetic acid overnight at 4° C. The solution is filtered through four layers of cheesecloth and is centrifuged as 12,000×g for two hours. The supernatant is precipitated with one-fifth volume of 30 g/dl NaCl and the pellet is collected by centrifugation at 4,000×g for thirty minutes. After two rinses with 5% g/dl NaCl and 0.6% acetic acid, the pellet is redissolved in 0.6% acetic acid. The solution is dialyzed against 1 mM HCl and is then sterilized by the additional of chloroform. A five ml aliquot is lyophilized to determine the concentration. Generally, 200 mg can be isolated from one rat tail. Collagen gel prepared by rapidly mixing the collage solution with 10×DMEM and incubating at 37° C. (U.S. Pat. No. 5,942,436).

Fibronectin

As a constituent of the extracellular matrix, fibronectin is important for allowing cells to attach to the matrix. Fibronectin influences both the growth and migration of cells. Normal fibroblasts in tissue culture secrete fibronectin and assemble it into a matrix that is essential to their adhesion and growth (U.S. Pat. No. 5,837,813).

The general structure of fibronectin is reviewed in Yamada, (1989) Current Opin. Cell Biol. 1, 956-963. The polypeptide is composed of a number of repeats, of which there are three kinds, type I, type II, and type III. The type I repeat is about 45 amino acids long and makes up the amino-terminal and carboxy-terminal ends of the polypeptide. Two 60 amino acid type II repeats interrupt a row of nine type I repeats at the amino-terminus of fibronectin. Finally, 15 to 17 type III repeats, each about 90 amino acids long, make up the middle of the polypeptide. Altogether, mature, i.e., processed, fibronectin contains nearly 2500 amino acid residues (U.S. Pat. No. 5,837,813).

Matrix assembly requires the binding of fibronectin to cell surfaces followed by assembly into fibrils, and stabilization of the fibrils by disulfide cross-linking. Several regions within fibronectin are required for the assembly process. The amino terminal 70 kDa region of fibronectin is known to bind to another molecule, the identity of which is unknown. (McKeown-Longo et al. (1985) J. Cell Biol. 100, 364-374; Mosher et al. (1991) Ann. N.Y. Acad. Sci. 614, 167-180).

The fibronectin molecule may be characterized as containing both heparin-binding regions and gelatin-binding regions. Another region considered to be involved in the fibronectin assembly process is the amino terminal 29 kDa heparin binding domain. Cells have been shown to organize fibronectin fragments into fibrils only when heparin-binding fragments and an RGD-containing cell binding domain were present simultaneously (Woods et al. (1988) Exp. Cell Res. 177, 272-283). The importance of the 29 kDa heparin-binding domain has been further underscored by the finding that recombinant fibronectin molecules lacking the 29 kDa region are not incorporated into extracellular matrix (Schwarzbauer, (1991) J. Cell Biol. 113, 1463-1473). Moreover, molecules composed only of the 29 kDa region, plus the carboxy-terminal half of fibronectin were efficiently incorporated into the extracellular matrix. In view of the above information, the role of the 29 kDa region appears to mediate the binding of fibronectin to the cell surface (U.S. Pat. No. 5,837,813).

Another region involved in matrix assembly is the RGD (arginine-glycine-aspartic acid)-containing cell binding domain of fibronectin. Monoclonal antibodies directed to the cell binding domain of fibronectin have been found to inhibit assembly of extracellular matrix (McDonald et al. (1987) J. Biol. Chem. 262, 2957-2967). In addition, two-monoclonal antibodies have been described that bind close to, but not directly to, the RGD site. These antibodies block the binding of cells to fibronectin and also block fibronectin matrix assembly (Nagai et al. (1991) J. Cell Biol. 114, 1295-1305).

The receptor that binds to the RGD site in fibronectin is, in most cells, the α₅β₁ integrin (Pierschbacher et al. (1984) Nature 309, 30-33). Accordingly, monoclonal antibodies directed against the α₁ and β₅ integrin subunits have also been found to inhibit fibronectin matrix assembly, as well as the binding of fibronectin to matrix assembly sites. Conversely, overexpression of the α₅β₁ integrin in CHO cells results in increased fibronectin matrix assembly. Taken together, these findings establish the importance of the interaction between fibronectin and the α₅β₁ integrin during matrix assembly.

Integrins themselves are heterodimeric transmenbrane receptors whose ligand-binding specificity is determined by the combination of α and β subunits. Of associations between the nine known β subunits and known α subunits, integrins α₅β₁, αIIbβ₃ and all or most αᵥ-containing integrins, but generally not others, recognize an arginine-glycine-aspartic acid (RGD) motif. Ligands for these RGD-binding integrins include a variety of extracellular matrix proteins such as fibronectin, vitronectin, osteopontin and collagens; plasma proteins such as fibrinogen and von Willebrand factor; cellular counter-receptors; the disintegrins; and viral proteins (U.S. Pat. No. 5,817,750).

Integrins are fundamental to processes of physical adhesion involving cell-cell or cell-matrix interactions and also can mediate signal transduction through their cytoplasmic domains. RGD-binding integrins function in biological processes including cell migration in development, wound healing and tissue repair, platelet aggregation and immune cell recognition. A role for these integrins also is implicated in a variety of pathologies including thrombosis, osteoporosis, tumor growth and metastasis, inflammation and diseases of viral etiology such as acquired immune deficiency syndrome. The physiological relevance of integrins is underscored by the observation that hereditary mutations can destroy RGD-binding activity and have pathological consequences resulting in, for example, the bleeding disorder, Glanzmann's thrombasthenia.

Peptides and protein fragments can be used to modulate the activities of RGD-binding integrins. One class of peptides that can act as competitors of RGD-binding activity includes peptides that contain the RGD motif or a functional equivalent of this motif. A second class of peptides includes those peptides that bind RGD-containing ligands through structures that function similarly to the integrin domain that contacts the RGD sequence. Peptides that structurally mimic the RGD-binding site in integrin β subunits, for example, can modulate the activity of RGD-binding integrins (e.g., cyclic RGD comprising peptides, U.S. Pat. No. 5,817,750).

A third region of-fibronectin has been shown to be involved in matrix assembly. A 56 kDa fragment from fibronectin, which contains the 40 kDa gelatin-binding domain, plus the first type III repeat has been found to inhibit the incorporation of exogenous fibronectin into the extracellular matrix (Chernousov et al. (1991) J. Biol. Chem. 266, 10851-10858). In addition, monoclonal antibodies that bind within this 56 kDa region were also found to block fibronectin matrix assembly.

Because of its role in the extracellular matrix, fibronectin is important in both normal and pathological tissues. The identification of additional regions of fibronectin involved in the assembly of extracellular matrix will provide additional means to control the matrix assembly process. Such control may be useful in many biologically and medically important situations, such as culturing cells and directing tissue regeneration, and ameliorating certain pathological conditions. The 3-D collagen-based constructs of the invention may comprise, in addition to or in place of intact fibronectin, digested fibronectin, fragments of fibronectin, or RGD-motif containing peptides.

Anti-Apoptotic Genes

An increasing number of genes and gene products have been implicated in apoptosis. One of these is bcl-2, which is an intracellular membrane protein shown to block or delay apoptosis. Overexpression of bcl-2 has been shown to be related to hyperplasia, autoimmunity and resistance to apoptosis, including that induced by chemotherapy (Fang et al. (1994) J. Immunol. 153, 4388-4398). A family of bcl-2-related genes has been described. All bcl-2 family members share two highly conserved domains, BH1 and BH2. Bcl-2 family members include, but are not limited to, A1, mcl-1, bcl-w, bax, bad, bak and bcl-x. A1, mcl-1, bcl-w and bcl-x1 (long form of bcl-x) are presently known to confer protection against apoptosis and are referred to as anti-apoptotic “bel-2-related” proteins.

In addition to bcl-2 related genes, several members of a new gene family of inhibitors of apoptosis related to the baculovirus LAP (Inhibitor of Apoptosis) gene (Birnbaum et al. (1994) J. Virol. 68, 2521-2528; Clem et al. (1994) Mol. Cell Biol. 14, 5212-5222) have been identified in Drosophila and mammalian cells (Duckett et al. (1996) EMBO J. 15, 2685-2694; Hay et al. (1995) Cell 83, 1253-1262; Liston et al. (1996) Nature 379, 349-353; Rothe et al. (1995) Cell 83, 1243-1252; Roy et al. (1995) Cell 80, 167-178). These molecules are highly conserved evolutionarily; they share a similar architecture organized in two or three approximately 70 amino acid amino terminus Cys/His baculovirus IAP repeats (BIR) and by a carboxy terminus zinc-binding domain, designated RING finger (Duckett et al. (1996) EMBO J. 15, 2685-2694; Hay et al. (1995) Cell 83, 1253-1262; Liston et al. (1996) Nature 379, 349-353; Rothe et al. (1995) Cell 83, 1243-1252; Roy et al. (1995) Cell 80, 167-178). Recombinant expression of IAP proteins blocks apoptosis induced by various stimuli in vitro (Duckett et al. (1996) EMBO J. 15, 2685-2694; Liston. et al. (1996) Nature 379, 349-353) and promotes abnormally prolonged cell survival in the developmentally-regulated model of the Drosophila eye, in vivo (Hay et al. (1995) Cell 83, 1253-1262).

Survivin is a recently identified gene encoding a structurally unique IAP apoptosis inhibitor. Survivin is a 16.5 kDa cytoplasmic protein containing a single BIR, and a highly charged carboxyl-terminus coiled-coil region instead of a RING finger, which inhibits apoptosis induced by growth factor (IL-3) withdrawal when transferred in B cell precursors (Ambrosini et al. (1997) Nature Med. 3, 917-921).

U.S. Pat. No. 6,015,687 (issued Jan. 18, 2000) discloses cdn-1 and cdn-2 as two new anti-apoptotic agents and homologs of Bcl-2. Rothe et al. (1995) Cell 83, 1243-1252 reports that the TNFR2-TRAF signaling complex contains two proteins related to baculoviral inhibitor of apoptosis proteins. U.S. Pat. No. 6,001,992 describes identification and cloning of two FADD-like anti-apoptotic molecules that regulate Fas/TNFR1- or UV-induced apoptosis.

The zinc finger protein A20 is a TNF-induced primary response gene that has been shown to inhibit TNF-induced apoptosis (Heyninck et al. (1999) Anticancer Res. 19, 2863-2868). Human and rat islets can be induced to rapidly express the anti-apoptotic gene A20 after interleukin-1 (IL-1) beta activation (Grey et al. (1999) J. Exp. Med. 190, 1135-1146). In A20 cells, Fas signaling may trigger both ICE activation and Bcl-x and Bcl-2 down-regulation (Bras et al. (1997) J. Immunol. 159, 3168-3177).

Caspases

Caspases are cysteine proteases that cleave after aspartic residues. Several members of the family have been implicated as key regulators of programmed cell death or apoptosis (Alnemri, (1997) J. Cell. Biochem. 64, 33-42 and Henkart, (1996) Immunity 4, 195-201). The pro-apoptotic caspases can be divided into two groups: those with a large prodomain such as ICH-1 (caspase-2), Mch4 (caspase-10), Mch5/MACH/FLICE (caspase-8) and Mch6/ICE-Lap-6 (caspase-9) and those with a small prodomain such as CPP32/YAMA/Apopain (caspase-3), Mch2 (caspase-6) and Mch3/ICE-Lap-3 (caspase-7).

Caspases with large prodomains are probably the most upstream caspases. They are recruited by several death-signaling receptors that-belong to the TNFR family, through interactions of their prodomain with the receptor-interacting adaptor molecules FADD/Mort1 or CRADD/RADD. For example, the prodomains of Mch4 and Mch5 contain two tandem regions that show significant homology with the N-terminal death effector domain (DED) of FADD. Engagement of Fas/TNFR1 results in recruitment of FADD to the receptor complex, which presumably triggers activation of the caspase apoptotic pathway through interaction of its DED with the corresponding motifs in the prodomain of Mch5 and probably Mch4, CRADD presumably functions like FADD by recruiting ICH-1 to the Fas/TNFR1 complex, through interaction of its N-terminal domain with the corresponding motif in the prodomain of ICH-1. Thus, the prodomains of caspases function to physically link the death receptors to the downstream caspase activation pathway.

Caspases can be controlled in two ways. The: processing and activation of a caspase can be regulated by anti-apoptotic factors such- as FADD, Bcl-2 family members, and IAPs and by modulators such as APAF-1 and FLIP. Active caspases can be controlled by a variety of inhibitors that directly interact with the protease. Ekert et al. (1999) Cell Death Differ. 6, 1081-1086 reviews caspases inhibitors that have been recently developed both as research tools and as pharmaceutical agents to inhibit cell death in vivo.

An example of a caspase inhibitor is CBZ-Val-Ala-Asp-fluoromethylketone (zVAD-fmk). Johnson et al. (1999) J. Biol. Chem. 274, 18552-18558 reports that Bcl-2 cleavage in response to TNF-α is inhibited by caspase inhibitor zVAD-fmk. Johnson et al. (1999) also shows that Bcl-2 cooperates with caspase inhibition to block TNF-α induced cell death.

The loop domain of Bcl-2-is cleaved at Asp34 by caspase-3 (CPP32) in vitro, in cells overexpressing caspase-3, and after induction of apoptosis by Fas ligation and interleukin-3 withdrawal. However mutations at amino acids 31 or 34 of the Bcl-2 sequence lead to non-cleavable Bcl-2 protein (Cheng et al. (1997) Science 278, 1966-1968).

The present invention discloses the use of Bcl-2 mutants. The preferred mutant is the D34A Bcl-2 (mutation of Aspartic Acid to Alanine at position 34). In place of, or in addition to the D34A form of Bcl-2, other anti-apoptotic proteins such as those described in the preceding sections may be transduced into EC before the cells are incorporated into the 3-D collagen-based construct of the invention. Said other anti-apoptotic proteins include but are not limited to “Bcl-2 related” proteins, the D31A form of Bcl-2, IAP-related proteins (for example, survivin) and A20.

The sequence of the preferred anti-apoptotic protein, D34A Bcl-2, is described in Cheng et al. (1997) Science 278, 1966-1968. The vector modified to carry and express the DNA encoding D34A Bcl-2 may also carry and express the DNA encoding a second protein of interest, including but not limited to the anti-apoptotic proteins described above, or other proteins that might modulate the processes of vascularization or vascular remodeling.

Recombinant DNA

The present invention utilizes recombinant DNA (rDNA) molecules that contain a coding sequence. As used herein, a rDNA molecule is a DNA molecule that has been subjected to molecular manipulation in situ. Methods for generating rDNA molecules are well known in the art, for example, see Sambrook et al. (1989) Molecular Cloning: A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extra-chromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 (BioRad Laboratories), pPL and pKK223. (Pharmacia).

Expression vectors compatible with eukaryotic cells, preferably those compatible with vertebrate cells such as kidney cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies), pTDT1 (ATCC), the vector pCDM8 described herein, and the like eukaryotic expression vectors. Vectors may be modified to include cell specific promoters if needed.

Eukaryotic cell expression vectors used to construct the rDNA molecules utilized in the present invention may further include a selectable marker that is effective in an eukaryotic. cell, preferably a drug resistance selection marker. A preferred drug resistance marker is the gene whose expression results in neomycin resistance, i.e., the neomycin phosphotransferase (neo) gene. (Southern et al. (1982) J. Mol. Appl. Genet., 1, 327-341) Alternatively, the selectable marker can be present on a separate plasmid, and the two vectors are introduced by co-transfection of the host cell, and selected by culturing in the appropriate drug for the selectable marker.

The present invention further utilizes host cells transformed with a nucleic acid molecule that encodes a protein. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells (CCL61, ATCC), NIH Swiss mouse embryo cells (NIH3T3) (CRL1658, ATCC), baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule is accomplished by well known methods that typically depend on the type of vector used and host system employed. With regard to transformation of prokaryotic host cells, electroporation and salt treatment methods are typically employed, see, for example, Cohen et al. (1972) Proc. Natl. Acad. Sci. USA 69, 2110-2114; and Sambrook et al., (1989) Molecular Cloning: A Laboratory Mammal, Cold Spring Harbor Laboratory Press. With regard to transformation of vertebrate cells with vectors containing rDNAs, electroporation, cationic lipid or salt treatment methods are typically employed, see, for example, Graham et al. (1973) Virol. 52, 456-467; Wigler et al. (1979) Proc. Natl. Acad. Sci. USA 76, 1373-1376.

Successfully transformed cells, i.e., cells that contain a rDNA molecule, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern (1975) J. Mol. Biol. 98, 503-517 or the proteins produced from the cell assayed via an immunological method.

In general terms, the production of a recombinant form of a protein typically involves the following steps:

First, a nucleic acid molecule is obtained that encodes a protein of interest. If the encoding sequence is uninterrupted by introns, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with any specific nucleic acid molecules to produce recombinant protein.

Vectors

A variety of vectors may be used as gene transfer vehicles, including viral vectors derived from retroviruses, adenoviruses, adeno-associated viruses (AAV) and lentiviruses. Such vectors may be modified to carry one or more genes of interest operably linked to control sequences. The present invention discloses the use of retroviral vectors, modified to encode and express Bcl-2, preferably the D34A form of Bcl-2. Such vectors may also be modified to encode and express a second gene of interest, for example VEGF or VEGF receptor or angiopoietin-1, or any of the anti-apoptotic proteins described above.

Retroviral Vectors

Replication-defective retroviral vectors as gene transfer vehicles provide the foundation for human gene therapy. Retroviral vectors are engineered by removing or altering all viral genes so that no viral proteins are made in cells infected with the vector and no further virus spread occurs. The development of packaging cell lines which are required for the propagation of retroviral vectors were the most important step toward the reality of human gene therapy. The foremost advantages of retroviral vectors for gene therapy are the high efficiency of gene transfer and the precise integration of the transferred genes into cellular genomic DNA. However, major disadvantages are also associated with retroviral vectors, namely, the inability of retroviral vectors to transduce non-dividing cells and the potential insertional mutagenesis. The construction of a Bcl-2 retroviral vector and the delivery of retroviral particles using an amphotropic packaging cell line are described in U.S. Pat. No. 6,027,721.

Adenoviruses

Human adenoviruses have been developed as live viral vaccines and provide another alternative for in vivo gene delivery vehicles for human gene therapy (Graham & Prevec (1992) New Approaches to Immunological Problems, Ellis (ed), Butterworth-Heinemann, 363-390; Rosenfeld et al. (1991) Science 252, 431-434; Rosenfeld et al. (1992) Cell 68, 143-155; Ragot et al. (1993) Nature 361, 647-650). The features which make recombinant adenoviruses potentially powerful gene delivery vectors have been extensively reviewed (Berkner (1988) Biotechniques 6, 616-629; Kozarsky et al. (1993) Curr. Opin. Genet. Dev. 3, 499-503). Briefly, recombinant adenoviruses can be grown and purified in large quantities and efficiently infect a wide spectrum of dividing and non-dividing mammalian cells in vivo.

Moreover, the adenoviral genome may be manipulated with relative ease and accommodate very large insertions of DNA. The first generation of recombinant adenoviral vectors currently available have a deletion in the viral early gene region 1 (herein called E1 which comprises the E1a and E1b regions from genetic map units 1.30 to 9.24) which for most uses is replaced by a transgene. A transgene is a heterologous or foreign (exogenous) gene that is carried by a viral vector and transduced into a host cell. Deletion of the viral E1 region renders the recombinant adenovirus defective for replication and incapable of producing infectious viral particles in the subsequently infected target cells (Berkner (1988) Biotechniques 6, 616-629). The ability to generate E1-deleted adenoviruses is based on the availability of the human embryonic kidney packaging cell line called 293. This cell line contains the E1 region of the adenovirus which provides the E1 region gene products lacking in the E1-deleted virus (Graham et al. (1972) J. Gen. Virol. 36, 59-72). However, the inherent flaws of current first generation recombinant adenoviruses have drawn increasing concerns about its eventual usage in patients. Several recent studies have shown that E1 deleted adenoviruses are not completely replication incompetent (Rich (1993) Hum. Gene. Ther. 4, 461-476; Engelhardt et al. (1993) Nature Genet. 4, 27-34). Three general limitations are associated with the adenoviral vector technology. First, infection both in vivo and in vitro with the adenoviral vector at high multiplicity of infection (moi) has resulted in cytotoxicity to the target cells, due to the accumulation of penton protein, which is itself toxic to mammalian cells. Second, host immune responses against adenoviral late gene products, including penton protein, cause the inflammatory response and destruction of the infected tissue which received the vectors (Yang et al (1994) Proc. Natl. Acad. Sci. USA 91, 4407-4411). Lastly, host immune responses and cytotoxic effects together prevent the long term expression of transgenes and cause decreased levels of gene expression following subsequent administration of adenoviral vectors (Mittal et al. (1993) Virus Res. 28, 67-90).

In view of these obstacles, further alterations in the adenoviral vector design are required to cripple the ability of the virus to express late viral gene proteins, decreasing host cytotoxic responses and the expectation of decreasing host immune response. Engelhardt et al. recently constructed a temperature sensitive (ts) mutation within the E2A-encoded DNA-binding protein (DBP) region of the E1-deleted recombinant adenoviral vector (Engelhardt et al. (1994) Proc. Natl. Acad. Sci. USA 91, 6196-6200) which fails to express late gene products at non-permissive temperatures in vitro. Diminished inflammatory responses and prolonged transgene expression were reported in animal livers infected by this vector (Engelhardt et al. (1994) Proc. Natl. Acad. Sci. USA 91, 6196-6200).

However, the ts DBP mutation may not give rise to a full inactive gene product in vivo, and therefore be incapable of completely blocking late gene expression. Further technical advances are needed that would introduce a second lethal deletion into the adenoviral E1-deleted vectors to completely block late gene expression in vivo. Novel packaging cell lines that can accommodate the production of second (and third) generation recombinant adenoviruses rendered replication-defective by the deletion of the E1 and E4 gene regions hold the greatest promise towards the development of safe and efficient vectors for human gene therapy (U.S. Pat. No. 5,872,005).

Transgenic Animals and Transgenic Animal Cells, Tissues, Organs

The term “animal” as used herein includes all vertebrate animals, except humans. It also includes an individual animal in all stages of development, including embryonic and fetal stages. A “transgenic animal” is an animal containing one or more cells bearing genetic information received, directly or indirectly, by deliberate genetic manipulation at a subcellular level, such as by microinjection or infection with recombinant virus. This introduced DNA molecule may be integrated within a chromosome, or it may be extra-chromosomally replicating DNA. The term “germ cell-line transgenic animal” refers to a transgenic animal in which the genetic information was introduced into a germ line cell, thereby conferring the ability to transfer the information to offspring. If such offspring in fact possess some or all of that information, then they, too, are transgenic animals. Transgenic animals containing mutant, knock-out, modified genes or gene constructs to over-express or conditionally express a gene corresponding to the cDNA sequences of caspase-resistant Bcl-2 or related sequences are encompassed in the invention.

The information may be foreign to the species of animal to which the recipient belongs, foreign only to the particular individual recipient, or genetic information already possessed by the recipient. In the last case, the introduced gene may be differently-expressed compared to the native endogenous gene. The genes may be obtained by isolating them from genomic sources, by preparation of cDNA from isolated RNA templates, by directed synthesis, or by some combination thereof.

To be expressed, a gene should be operably linked to a regulatory region. Regulatory regions, such as promoters, may be used to increase, decrease, regulate or designate to certain tissues or to certain stages of development the expression of a gene. The promoter need not be a naturally occurring promoter. The “transgenic non-human animals” of the invention are produced by introducing “transgenes” into the germline of the non-human animal. The methods enabling the introduction of DNA into cells are generally available and well-known in the art. Different methods of introducing transgenes could be used. Generally, the zygote is the best target for microinjection. In the mouse, the male pronucleus reaches the size of approximately twenty microns in diameter, which allows reproducible injection of one to two picoliters of DNA solution. The use of zygotes as a target for gene transfer has a major advantage. In most cases, the injected DNA will be incorporated into the host gene before the first cleavage (Brinster et al. (1985) Proc. Natl. Acad. Sci. USA 82, 4438-4442). Consequently, nearly all cells of the transgenic non-human animal will carry the incorporated transgene. Generally, this will also result in the efficient transmission of the transgene to offspring of the founder since 50% of the germ cells will harbor the transgene. Microinjection of zygotes is a preferred method for incorporating transgenes in practicing the invention.

Retroviral infection can also be used to introduce a transgene into a non-human animal. The developing non-human embryo can be cultured in vitro to the blastocyst stage. During this time, blastomeres may be targets for retroviral infection. Efficient infection of the blastomeres is obtained by enzymatic treatment to remove the zona pellucida. The viral vector system used to introduce the transgene is typically a replication-defective retrovirus carrying the transgene (Jahner et al. (1985) Proc. Natl. Acad. Sci. USA 82, 6927-6931; Van der Putten et al. (1985) Proc. Natl. Acad. Sci. USA 82, 6148-6152). Transfection is easily and efficiently obtained by culturing the blastomeres on a monolayer of virus-producing cells (Van der Putten et al. (1985) Proc. Natl. Acad. Sci. USA 82, 6148-6152; Stewart et al. (1987) EMBO J. 6, 383-388). Alternatively, infection can be performed at a later stage. Virus or virus-producing cells can be injected into the blastocoele (Jahner et al. (1982) Nature 298, 623-628). Most of the founder animals will be mosaic for the transgene since incorporation occurs only in a subset of the cells which formed the transgenic non-human animal. Furthermore, the founder animal may contain retroviral insertions of the transgene at a variety of positions in the genome; these generally segregate in the offspring. In addition, it is also possible to introduce transgenes into the germ line, albeit with low efficiency, by intrauterine retroviral infection of the midgestation embryo (Jahner et al. (1982) Nature 298, 623-628).

A third type of target cell for transgene introduction is the embryonal stem cell (ES). ES cells are obtained from pre-implantation embryos cultured in vitro (Evans et al. (1981) Nature 292, 154-156; Bradley et al. (1984) Nature 309, 255-256; Gossler et al. (1986) Proc. Natl. Acad. Sci. USA 83, 9065-9069). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal.

The methods for evaluating the presence of the introduced DNA as well as its expression are readily available and well-known in the art. Such methods include, but are not limited to DNA (Southern) hybridization to detect the exogenous DNA, polymerase chain reaction (PCR), polyacrylamide gel electrophoresis (PAGE) and Western blots to detect DNA, RNA and protein. The methods include immunological and histochemical techniques to detect expression of a gene.

As used herein, a “transgene” is a DNA sequence introduced into the germline of a non-human animal by way of human intervention such as by way of the Examples described below. The nucleic acid sequence of the transgene may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

As discussed above, a “vector” is any means for the transfer of a nucleic acid into a host cell. Preferred vectors are plasmids and viral vectors, such as retroviruses. Viral vectors may be used to produce a transgenic animal according to the invention. Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents.

Preferably, the replication defective virus retains the sequences of its genome which are necessary for encapsidating the viral particles. The retroviruses are integrating viruses which infect dividing cells. The retrovirus genome includes two LTRs, an encapsidation sequence and three coding regions (gag, pol and env). The construction of recombinant retroviral vectors has been described (see, for example, Bernstein et al. (1985) Genet. Eng. 7, 235-236; McCormick (1985) Biotechnol. 3, 689-691). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (murine Moloney leukemia virus), MSV (murine Moloney sarcoma virus), HaSV (harvey sarcoma virus); SNV (spleen necrosis virus); RSV (Rous sarcoma virus) and Friend virus;

In general, in order to construct recombinant retroviruses containing a nucleic acid sequence, a plasmid is constructed which contains the LTR, the encapsidation sequence and the coding sequence. This construct is used to transfect a packaging cell line, which cell line is able to supply in trans the retroviral functions which are deficient in the plasmid. In general, the packaging cell lines are thus able to express the gag, pol and env genes. Such packaging cell lines have been described in the prior art, in particular the cell line PA317 (U.S. Pat. No. 4,861,719); the PsiCRIP cell line (WO 90/02806) and the GP+envAm-12 cell line (WO 89/07150). In addition, the recombinant retroviral vectors can contain modifications within the LTR for suppressing transcriptional activity as well as extensive encapsidation sequences which may include a part of the gag gene (Bender et al. (1987) J. Virol. 61, 1639-1646). Recombinant retroviral vectors are purified by standard techniques known to those having ordinary skill in the art.

In one aspect the nucleic acid encodes antisense RNA molecules. In this embodiment, the nucleic acid is operably linked to suitable regulatory regions (discussed above) enabling expression of the nucleic acid sequence, and is introduced into a cell utilizing, preferably, recombinant vector constructs, which will express the antisense nucleic acid once the vector is introduced into the cell. Examples of suitable vectors includes plasmids, adenoviruses, adeno-associated viruses (see, for example, U.S. Pat. Nos. 4,797,368 & 5,139,941), retroviruses (see above), and herpes viruses. For delivery of a therapeutic gene the vector is preferably an adenovirus.

Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type two or type five human adenoviruses (Ad 2 or Ad 5) or adenoviruses of animal origin (see WO 94/26914). Those adenoviruses of animal origin which can be used within the scope of the present invention include adenoviruses of canine, bovine, murine, ovine, porcine, avian, and simian origin.

The replication defective recombinant adenoviruses according to the invention can be prepared by any technique known to the person skilled in the art. In particular, they can be prepared by homologous recombination between an adenovirus and a plasmid which carries, inter alia, the DNA sequence of interest. The homologous recombination is effected following cotransfection of the said adenovirus and plasmid into an appropriate cell line. The cell line which is employed should preferably (i) be transformable by the said elements, and (ii) contain the sequences which are able to complement the part of the genome of the replication defective adenovirus, preferably in integrated form in order to avoid the risks of recombination. Recombinant adenoviruses are recovered and purified using standard molecular biological techniques, which are well known to one of ordinary skill in the art.

A number of recombinant or transgenic mice have been produced, including those which express an activated oncogene sequence (U.S. Pat. No. 4,736,866); express Simian SV 40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al. (1996) Genetics 143, 1753-1760) or are capable of generating a fully human antibody response (Zou et al. (1993) Science 262, 1271-1274).

While mice and rats remain the animals of choice for most transgenic experimentation, in some instances it is preferable or even necessary to use alternative animal species. Transgenic procedures have been successfully utilized in a variety of non-murine animals, including sheep, goats, chickens, hamsters, rabbits, cows and guinea pigs (see Aigner et al. (1999) Biochem. Biophys. Res. Commun. 257, 843-850; Castro et al. (1999) Genet. Anal. 15, 179-187; Brink et al. (2000) Theriogenology 53, 139-148; Colman (1999) Genet. Anal. 15, 167-173; Eyestone (1999) Theriogenology 51, 509-517; Baguisi et al. (1999) Nat. Biotechnol. 17, 456-461; Prather et al. (1999) Theriogenology 51, 487-498; Pain et al. (1999) Cells Tissues Organs 165, 212-219; Fernandez et al. (1999) Indian J. Exp. Biol. 37, 1685-1092; U.S. Pat Nos. 5,908,969; 5,792,902; 5,892,070 & 6,025,540).

In Situ Transformation

In another embodiment, the present invention relates to the delivery of DNA into individual cells of an animal. For examples of in situ or in vivo transfection and transduction methods, see, for example, Ram et al. (1993) Cancer Research 53, 83-88; Logeart et al. (2000) Hum. Gene Ther. 11, 1015-1022; Widera et al. (2000) J. Immunol. 64, 4635-4640.

Gene Therapy

In a further embodiment, the present invention also relates to methods of removing cells from the mammal, introducing into the cells a DNA molecule encoding a protein of interest, and reintroducing the cells into the mammal under conditions such that the DNA molecule is expressed. For examples of such methods, see, for example, U.S. Pat Nos. 6,068,983; 6,066,624 & 6,068,837; Miller (1990) Blood 76, 271-278; Selden et al. (1987) New Eng. J. Med. 317, 1067-1076.

Tissue and Organ Transplantation

“Transplantation” as used herein, generally refers to the process by which a body part, organ, tissue or cell is transferred from one organism to another organism or transferred to an organism from an artificial source such as an organ or tissue harvested from cell or tissue culture systems. “Graft” as used herein, generally refers to a body part, organ, tissue, or cells. Grafts may consist of organs such as liver, kidney, heart or lung; body parts such as bone or skeletal matrix; tissue such as skin, intestines, endocrine glands; or progenitor stem cells of various types. For general information on transplantation and grafting, see, for example, Flye (1995) Atlas of Organ Transplantation, Saunders.

There continues to bean extreme shortage of organs for transplantation. For example, kidney transplantation is largely dependant upon the availability of organs retrieved from heart-beating cadaver donors. A large and as yet untapped source of organs for transplantation are accident victims who succumb at the site of an injury and those having short post-trauma survival times. These accident victims are not used as organ donors because of the ischemic damage. Likewise, older potential donors are often considered borderline because of questions relating to organ function.

Despite significant advances in understanding of tissue typing and immunosuppression and the availability of better immunosuppressive agents, acute rejection remains a serious clinical problem. As is well known, the use of immunosuppressive agents to avoid rejection of such grafts is also accompanied by a host of problems.

A primary function of the immune response is to discriminate self from non-self antigens and to eliminate the latter. The immune response involves complex cell to cell interactions and depends primarily on three major cell types: thymus derived (T) lymphocytes, bone marrow derived (B) lymphocytes, and macrophages. The immune response is mediated by molecules encoded by the major histocompatibility complex (MHC). The two principal classes of MHC molecules, Class I and Class II, each comprise a set of cell surface glycoproteins (Stites & Terr (1991) Basic and Clinical Immunology, Appelton & Lang). MHC Class I molecules are found on virtually all somatic cell types, although at different levels in different cell types. In contrast, MHC Class II molecules are normally expressed only on a few cell types, such as lymphocytes, macrophages and dendritic cells. Soluble MHC class I molecules have been shown to reduce rejection of allogeneic transplanted tissue in rats (Geissler et al. (1997) Transplantation 64, 782-786).

Antigens are presented to the immune system in the context of Class I or Class II cell surface molecules; CD4⁺ helper T-lymphocytes recognize antigens in association with Class II MHC molecules, and CD8⁺ cytotoxic T lymphocytes (CTL) recognize antigens in association with Class I gene products. It is currently believed that MHC Class I molecules function primarily as the targets of the cellular immune response, whereas the Class II molecules regulate both the humoral and cellular immune response (Klein & Gutze (1977) Major Histocompatibility Complex, Springer Verlag; Roitt (1984) Triangle 23, 67-76; Unanue (1984) Ann. Rev. Immunol. 2, 295-428). MHC Class I and Class II molecules have been the focus of much study with respect to research in autoimmune diseases because of their roles as mediators or initiators of the immune response. MHC-Class II antigens have been the primary focus of research in the etiology of autoimmune diseases, whereas MHC-Class I has historically been the focus of research in transplantation rejection.

T lymphocytes are known to play a key role in allograft rejection. Activated T lymphocytes have been identified as IL-2 receptor bearing cells. Several murine anti-IL-2 receptor antibodies have been administered in clinical trials for the prophylaxis and treatment of allograft rejection. See, for example, Carpenter et al. (1989) Am. J. Kid. Dis. 14, 54-57; Kirkman et al. (1991) Transplantation 51, 107-113 (anti-Tac); Soulillou et al. (1987) Lancet 1, 1339-1342; Soulillou et al. (1990) New Eng. J. Med. 322, 1175-1182 (33B3.1); Herve et al. (1990) Blood 75, 1017-1023 (B-B10); Nashan et al. (1996) Transplantation, 61, 546-554.

Methods of inactivating T cells, preferably thymic or lymph node T cells, can be used with other methods of inducing tolerance in which the inactivation of thymic or lymph node T cells is desirable. For example, anti-thymic or lymph node T cell methods can be used with: methods which use the implantation of a xenogeneic thymic graft to induce tolerance; methods of increasing the level of the activity of a tolerance promoting or Graft versus Host Disease (GvHD) inhibiting cytokine or decreasing the level of activity of a tolerance inhibiting or GvHD promoting cytokine; methods of using cord blood cells to induce; and the methods for inducing tolerance disclosed in Sykes & Sachs (1994) Immunol Rev. 141, 245-276. An immunosuppressive agent generally refers to an agent capable of inactivating thymic or lymph node T cells. Such agents include, but are not limited to, chemical agents, e.g., a drug, which, when administered at an appropriate dosage, results in the inactivation of thymic or lymph node T cells. Examples of such agents are cyclosporine, FK-506, and rapamycin. Anti-T cell antibodies, because they are comparatively less effective at inactivating thymic or lymph node T cells, are not preferred for use as agents. An agent should be administered in sufficient dose to result in significant inactivation of thymic or lymph node T cells which are not inactivated by administration of an anti-T cell antibody, e.g., an anti-ATG preparation. Putative agents, and useful concentrations thereof, can be prescreened by in vitro or in vivo tests, e.g., by administering the putative agent to a test animal, removing a sample of thymus or lymph node tissue, and testing for the presence of active T cells in an in vitro or in vivo assay. Such prescreened putative agents can then be further tested in transplant assays.

Attempts to transplant organ tissues into genetically dissimilar hosts without immunosuppression are generally defeated by the immune system of the host. A successful cell or tissue transplant must be coated with a coating which will prevent its destruction by a host's immune system, which will prevent fibrosis, and which will be permeable to and allow a free diffusion of nutrients to the coated transplant and removal of the secretory and waste products from the coated transplant. Attempts to provide effective protective barrier coatings to isolate the transplant tissues from the host immune system have not generally proven to be medically practical because the coating materials were incompatible with the host system or were otherwise unsuitable. The encapsulation or coating processes developed previously have not yielded reproducible coatings having the desired porosity and thickness required for the transplanted tissue to have a long and effective functional life in the host.

A primary problem with these coated cell or tissue transplants is that they are treated as foreign objects in the host's body and subject to immune rejection or destruction. For additional information on various methods used in an attempt to prevent transplanted organ rejection, see, for example, U.S. Pat. Nos. 5,728,721; 5,843,425; 5,871,950; 5,914,314 & 6,013,256.

To protect transplants from destruction by the immune response of the host animal, various attempts have been made to create a protective barrier between the transplant tissue or cells and the immunological components of the host's system. One approach is to employ microencapsulation of erythrocyte hemolysate and urease in semi-permeable polyamide membranes (see, for example, Science (1964) 146, 524-525). However, these microcapsules did not survive for long when injected into the blood stream. Both the preparation of semi-permeable microencapsulated microbial cells and viable red blood cells, and also the possibility of using injections of encapsulated cells for organ replacement therapy (Acta Chem. Scand. (1966) 20, 2807-2812; Can. J. Physiol. Pharmacol. (1966) 44, 115-128).

Natural and Synthetic Skin

The loss of cutaneous material for reasons of traumatic or pathological origin is commonly resolved by the autotransplantation technique, using skin explants from donor areas. To cover larger areas these explants can be expanded by surgical methods such as the mesh grafting described by Mauchahal (1989) J. Plast. Surg. 42, 88-91. These methods give positive results only with small-dimension lesions and patients with a satisfactory general health profile. If elderly patients or those in a state of serious decline are treated, unsatisfactory results are obtained and numerous problems arise, to the extent that such procedures cannot be used. In addition they do not allow a donor tissue expansion of more than ten-fold.

An important turning point in the treatment of these lesions by reconstructive surgery was the development of the technique involving the in vitro culture of keratinocytes (Rheinwald et al. (1975) Cell 6, 331-344), which allowed the in vitro expansion of these cultures, to obtain epidermic cell membranes potentially suitable for covering lesion areas. This technique has been widely used in clinical practice, mostly in the case of patients suffering from burns (Gallico et al. (1984) New Engl. J. Med. 311, 448-451), but numerous problems arose from its conception, such as the failure to take of some grafts, the fragility of the epithelial film and the consequent difficulty in its handling by the surgeon, the length of time required for obtaining sufficient quantities of epidermic cultures and the difficulty of obtaining donor areas of sufficient size from patients with large areas of damaged body surface. The in vitro epidermic cultures also require precise orientation to enable the graft to take, this being a particularly risky operation in view of the fragility of in vitro cultivated epidermic film.

A different approach to these problems is described by Yannas et al. (1982) Science 215, 174-176, who use dermic substitutes in the form of reabsorbable porous materials consisting of coprecipitates of collagen and glycosaminoglycans (GAG), in particular chondroitin-6-sulphate, covered by a thin silicone membrane film. The characteristic of these materials is that they comprise non-standardized pores intercommunicating in a manner similar to a sponge.

Zang et al. (1986) Burns 12, 540-543 propose a method, known as microskin grafting, consisting of auto-grafting very small skin portions, which then develop to merge into a single epithelium. With this method the maximum donor surface/coverable surface expansion ratio obtainable is 1:15.

Boyce et al. (1988) Surgery 103, 421-431 describe the use of membranes formed from collagen and GAG to promote on their surface the growth of keratinocytes, so reducing the surface porosity of the material. A continuous non-porous layer is also interposed to limit the epidermic culture development to the membrane surface. The possible antigenicity of these dermic substituents, which can result in rejection of the graft, has not yet been properly ascertained.

Prior approaches which have been used to develop a skin substitute can be divided into four broad categories, namely: homografts; modified dermal xenografts; synthetic polymeric structures; and, reconstituted collagen films.

The use of homografts in the treatment of massive burns is an accepted procedure at the present time. The source of the skin transplant may be a live donor or skin obtained from cadavers and preserved in a skin bank. The justification for the use of homografts is the necessity for reducing fluid loss, preventing infections, and reducing the area of scarring. In the absence of immunosuppressive agents, however, homografts are almost invariably rejected. Rejection is apparently mediated primarily by the interception of graft vascularization which accompanies the onset of the immune reaction. See, for example, Matter et al. (1971) Research in Burns, Hans Huber.

Efforts to modulate the immunogenicity of homografts by organ culture techniques have been attempted by several investigators. After a number of conflicting reports, the results of a definitive investigation of such procedures was reported by Ninnemann and Good who concluded that modification of antigens in the cultured tissue had not been demonstrated in such attempts (Ninnemann et al. (1974) Transplantation 18, 1-5).

An alternative approach to the use of homografts has been investigation of the possibility of modifying skin from animals. The basic goal of this approach is removal of those components in the dermis which elicit the production of host antibodies.

Other researchers pursued this approach by treating porcine dermis with trypsin to remove cellular and non-collagenous material (Oliver et al. (1972) Brit. J. Exp. Path. 53, 540-549). This resulted in a graft material which was primarily insoluble collagen cast in the original morphology of the dermis, with a negligible level of antigenicity. The modified dermal collagen thus obtained was grafted onto full thickness excised skin wounds in the pig and its fate compared to that of autografts and homografts of untreated dermis. The autografts behaved in the normal manner, described previously by Henshaw & Miller (1965) Arch. Surg. 91, 658-670). The untreated homografts were dead by day five, with mononuclear cells present, and had begun to degenerate at the base by day ten. By day twenty, the rejection of the homografts was substantially complete. With treated dermal collagen grafts, the lower part of the graft was repopulated with capillaries and fibroblasts by day five, while epidermal migration took place through the graft. Basophilic collagen lysis of the graft collagen started near day five and was associated with infiltration of granulation tissue which progressively replaced collagen in the presence of multinuclear giant cells. By day twenty, the grafts were substantially replaced by granulation tissue and behaved like open wounds. This emphasizes the necessity for increasing the resistance of native collagen to lysis.

As a result of these experiments, four requirements for a successful graft were established: (1) dermal collagen fibers should persist unaltered for a long period, providing an essential structural framework for the reformation of the vascular and cellular elements of tissue; (2) the graft should not evoke foreign body reaction, which leads to eventual destruction of the newly cellularized graft; (3) the graft should provide a suitable dermal bed for the growth and development of normal epidermis; and (4) the graft should suppress the formation of granulation tissue.

A third approach involves the use of synthetic polymeric structures. The literature is replete with references to the investigation of polymeric materials for a variety of biomedical applications including skin substitutes or temporary wound dressings. This is not surprising in view of the polymer scientist's capability of incorporating almost any set of physical and chemical (but, as yet, few biological) requirements into a polymeric structure. The investigations into the utility of polymeric films as skin replacements have, thus far, eliminated a considerable number of candidate materials but have resulted in useful insights into the requirements for a satisfactory skin replacement. For example, the use of velour structures resulted in improved adhesion to tissue, and the development of methods of preparation of so-called biocompatible polymers with controlled pore size improved the possibility of synthesizing materials capable of inducing cellular migration and proliferation into the graft. See, for example, Hall et al. (1967) Biomed. Mat. Res. 1, 187-189; Wilkes et al. (1973) Biomed. Mat. Res. 7, 541-542, respectively.

Another promising approach involved polymerization of crosslinked polymers in the hydrogel form, thus providing added capability for encouraging cellular ingrowth and vascularization (Hubacek et al. (1967) Biomed. Mat. Res. 1, 387-389). The use of synthetic polymers in skin replacement has not so far led to solution of the problem, however, due mainly to the high incidence of infection and the inability of the materials evaluated up to now to encourage vascularization and epithelialization.

Since the major constituent of normal skin is collagen, a logical approach to the development of a skin substitute would involve study of the fate of reconstituted collagen structures when placed in contact with living tissue. This approach was used by a number of investigators using the general procedure of extracting the collagen from animals, purifying it to various degrees and converting it to films or other structures that were used as wound dressings or implanted in living tissue to determine their in vivo fate. Earlier work in this area demonstrated that collagen per se evokes a chronic inflammatory response with subsequent resorption of the implant (Pullinger et al. (1942) J. Path. Bact. 34, 341-342). Other researchers were able to show that the rate of resorption of collagen could be reduced by controlled crosslinking with formaldehyde. They were also able to show that the immune response to reconstituted collagen implants was minimal (Grillo et al. (1962) J. Surg. Res. 2, 69-70).

Enzymatically modified collagen has been prepared and evaluated by Rubin and Stenzel who showed that this treatment does not evoke as much cellular response as the untreated material. The explanation for this variation in behavior is that the enzyme used (proctase) effectively removes the telopeptides from the collagen molecule without destroying the native molecular structure. Stark & Aggarwal (1969) Biomaterials, Plenum Press. The use of reconstituted collagen sheets has not eliminated the problems of lysis, infection and prevention of tissue ingrowth and vascularization encountered by use of other approaches. For additional information on synthetic and artificial skin substitutes, see, for example, U.S. Pat. Nos. 4,051,848; 5,196,190; 5,658,331; 5,727,567 & 5,800,811.

Synthetic Skin

Tissue engineering involves the development of new materials or devices capable of specific interactions with biological tissues. Wound care was one of the first fields to see the benefit tissue engineering. In wound care, these materials may be based entirely on naturally occurring tissues and cells, or may be materials that combine synthetics, usually polymers, with biological layers. Both wound dressings and skin substitutes are now clinically available (Phillips (1998) Arch. Dermatol. 134, 344-349). The complexity of the materials depends on the end uses. Generally, synthetics made from polymeric materials such as Tegaderm® and Opsite® are used as wound dressings over relatively simple and shallow wounds or as coverings over more complex dressings. Their function is one of protection from water loss, drying and mechanical injury. More complex dressings vary from dermal replacements made of reconstituted collagen and chondroitan sulfate backed by a polymer layer such as Integra® to the complex Apligraft® that contains collagen and seeded cells. This last is designed as a complete skin replacement (also considered a skin equivalent or skin substitute) and was approved as a biomedical device by the U.S. Food & Drug Administration (FDA) in 1998. TransCyte® a nonliving wound covering was approved by the FDA in 1997 and FDA action on Dermagraft® which consists of living cells, is pending. Ultimately, engineered skin will contain all of the components necessary to modulate healing and provide the desired response: a wound closed with limited scar tissue that retains all of the characteristics of natural skin. (see Sefton & Woodhouse (1998) J. Cutan. Med. Surg. 3, 18-23).

An overview of the field of tissue engineering is available in Lanza et al. (1997) Principles Of Tissue Engineering, Pergamon Press; Patrick et al. (1998) Frontiers In. Tissue Engineering, Pergamon Press; Edington et al. (1992) BioTechnology 10, 855-860.

Three-dimensional culture has become important in the formation of skin equivalents having both a differentiated epidermis and underlying dermis. In vivo, epidermal cells (keratinocytes) adhere tightly to one another and form a multilayered sheet that rests on a basal lamina. The keratinocytes of the basal layer are relatively undifferentiated and proliferate steadily, releasing progeny into the upper layers. There cell division halts and terminal differentiation occurs. Given a suitable substratum, dissociated keratinocytes in culture will likewise proliferate and differentiate. Under appropriate culture conditions they will develop into a multilayered epithelium in which the proliferating cells form the basal layer adherent to the substratum and the differentiating cells are segregated into the upper layers, just as in normal skin.

Keratinocyte grafting can be used to treat acute traumatic and chronic non-healing wounds, however the keratinocyte sheets are fragile and often do not “take” clinically. Success is enhanced by pre-treating the wound bed with viable dermis (Myers et al. (1995) Am. J. Surg. 170, 75-83). Current approaches culture keratinocytes directly on dermal complexes. For example, Maruguchi created continuous keratinocytes layers on an artificial skin dermis by the air-liquid interface culture method. The keratinocytes proliferated well and differentiated properly on this matrix, with a histologic appearance similar to that of normal epidermis (Maruguchi et al. (1994) Plast. Reconstr. Surg. 93, 537-544). The artificial dermis was a fibroblast filled collagen “sponge”, formed by incubating fibroblasts within a pre-formed network of collagen fibers. Collagen sponge formation and the air-liquid interface culture method are known to those of ordinary skill in the art. For sample protocols refer to U.S. Pat. Nos. 6,051,425 & 5,945,101. Many variations of this approach have been tested, using keratinocytes layered over fibroblasts embedded in a various scaffolds. For example, pre-formed scaffolds have also been constructed as collagen foams or threads (U.S. Pat. No. 6,051,750) and of synthetic polymers (U.S. Pat. No. 5,770,417; Zacchi et al. (1998) J. Biomed. Mater. Res. 40, 187-194).

Simple equivalents of the human dermis may also be prepared in vitro without a pre-formed scaffold; by mixing normal human fibroblasts with a collagen solution and then allowing the combination to form a 3-D gel. In such living skin equivalents, as in the vascular bed constructs discussed above, the collagen lattice remains hydrated (i.e., as a gel, as opposed to a sponge which may be dehydrated at some stage) and is maintained under conditions which permit living cells to survive. Keratinocytes are layered on top and allowed to differentiate before transplantation (see, for example, U.S. Pat. No. 4,485,096; Bell et al. (1979) Proc. Natl. Acad. Sci. USA, 76, 1274-1278; Dubertret (1990) Skin Pharmacol. 3, 144-148).

In the early post-transplantation period, prior to host neovascularization, transplanted tissues are wholly dependent on diffusion for survival (Young et al. (1996) J. Bum Care Rehabil. 17, 305-310). The lack of a vascular plexus leads to greater time for vascularization compared with native skin autografts and contributes to graft failure. The clinical experience with synthetic skins indicates that the absence of early perfusion may significantly limit the success of engineered tissues, especially when implanted into compromised recipient beds (e.g., in diabetes, thermal bums, or venous leg ulcers) (Young et al. (1996) J. Bum Care Rehabil. 17, 305-310; Grey et al. (1998) J. Wound Care 7,324-325) or in hosts with impaired angiogenesis (e.g., the elderly).

To enhance vascularization, Supp et al. modified human keratinocytes to overexpress vascular endothelial growth factor (VEGF), a specific and potent mitogen for endothelial cells. Collagen-based cultured skin substitutes inoculated with human fibroblasts and factor-modified keratinocytes exhibited increased numbers of dermal blood vessels and decreased time to vascularization when grafted to full-thickness wounds on athymic mice (Supp et al. (2000) J. Invest. Dermatol. 114, 5-13). Others have genetically modified fibroblasts before incorporating them into a collagen scaffold to prolong the survival of implanted cells (Rosenthal et al. (1997) Anticancer Res 17, 1179-1186). Factors such as TGF-β have also been included in the collagen matrix to inhibit inflammatory processes while promoting angiogenesis and histogenesis (U.S. Pat. No. 5,800,811).

Black et al. described the 3-D co-culture of endothelial cells with fibroblasts and keratinocytes to generate an endothelialized tissue-engineered skin with capillary-like structures (Black et al. (1998) FASEB J. 12, 1331-1340). Growth on collagen gels also promotes the cell organization and capillary formation of microvascular endothelial cells in human skin (Nör et al. (1999) Am. J. Pathol. 154, 375-384). A method for growing human dermal microvascular endothelial cells in a simplified liquid growth medium, thereby facilitating interpretation of experimental results, has been described by Kraling et al. (1998) In Vitro Cell. Dev. Biol. Anim. 34, 308-315.

Synthetic vascular beds of the invention will be used to increase the extent of perfusion and thereby improve survival of transplanted tissue, such as synthetic skins. It has been proposed that newly formed capillary tubes of microvascular ECs in 3-D culture must be invested with pericytes to maintain their integrity. As disclosed herein, this level of maturation and inosculation with adjacent vascular beds has been observed upon in vivo implantation of 3-D EC cultures transduced with caspase-resistant Bcl-2. Therefore, incorporation of the 3-D constructs of the present invention into transplanted tissues is likely to greatly improve the clinical success of transplantation procedures.

Endothelial cells transduced with caspase-resistant Bcl-2 may be suspended in a dermal equivalent comprising a collagen matrix containing fibronectin. The dermal equivalent may alternatively, or in addition, contain other matrix components that may be utilized to enhance survival of incorporated cells, reduce immunogenicity, or enhance structural integrity of engineered skin. Examples of such additional matrix components include vitronectin, fibrin, laminin, and additional collagen subtypes types as well as proteoglycans such as dermatan sulfate.

The dermal equivalent may include cells other than endothelial cells, which may or may not be genetically modified. These cells will be added to improve the overall survival and engraftment of the constructs, as well as to add functionality. These cells may include, but are not limited to fibroblasts and smooth muscle cells. An alternative strategy is to use acellular human or porcine dermis as the matrix rather than a synthetic matrix. If this strategy is employed, endothelial and possibly other cell types will be allowed to grow into, rather than be initially suspended in, the matrix. Whatever matrix strategy is used, cultured keratinocytes may be placed on the surface of the constructs, and subjected to conditions that promote differentiation into a stratified epidermis, for example, as in the air-liquid interface method noted above.

Human acellular dermis has been used as a temporary skin substitute for a variety of clinical applications, including burns, surgical wounds, and chronic ulcers. Although acellular dermis appears to improve wound healing, it does not truly engraft, and is eventually sloughed. The most likely explanation for the lack of engraftment is that acellular dermis is avascular, and, consequently, inadequately perfused.

To overcome the shortcomings of the currently available skin equivalents, acellular dermis can be vascularized with human endothelial cell lined blood vessels using the compositions, constructs and methods of the present invention. Specifically, HUVEC may be used to seed acellular dermis, which become perfused when implanted into immunodeficient mice. Endothelial cells that are incorporated in acellular dermis or other skin substitutes can also be genetically manipulated by retroviral transduction. For example, overexpression of the survival gene Bcl-2 in the HUVEC can increase graft perfusion. Endothelial cells may also be genetically manipulated to improve resistance to graft rejection, improve drug delivery, or increase angiogenesis. The capacity for genetic manipulation of the cells incorporated in acellular dermis and other constructs, and the selective inclusion of different cells, should offer significant advantages over models using whole skin. There are, however, potential hazards to the incorporation of genetically modified endothelial cells into synthetic tissues intended for human use. First, the possibility of producing infectious retrovirus is a concern that has been significantly minimized, or even eliminated, by using a packaging cell system which can not incorporate viral replication genes into the vector Pear et al. (1993) Proc. Natl: Acad. Sci. USA 90, 8392-8396. Another concern is that the target cell may undergo malignant transformation. In vitro experiments (Zheng et al. (2000) J. Immunol. 164, 4665-4671) have shown that Bcl-2-transduced HUVEC show no evidence of transformation in culture, and we found no evidence of tumor formation or invasion of mouse tissue by Bcl-2-transduced cells in vivo. Furthermore, over expression of Bcl-2 by retroviral tansduction in a low grade vascular tumor model did not increase the occurrence of metastases, indicating that this modification does not have a further transforming effect. The safety of these manipulations will require further evaluation, but there are no indications to date that suppression of apoptosis in endothelium is by itself tumorogenic.

Identification of Differentially Expressed Genes and Proteins

This section describes methods for the identification of genes and gene products that are involved in vascular remodeling. “Differential expression” as used herein refers to both quantitative as well as qualitative differences in the temporal and/or tissue expression patterns of genes or proteins. Thus, a differentially expressed gene or protein may have its expression activated or completely inactivated in control versus experimental cells or conditions. Such a qualitatively regulated molecule will exhibit an expression pattern within a given tissue or cell type that is detectable in either control or experimental cells or conditions, but is not detectable in both. Alternatively a differentially expressed gene or protein may have its expression modulated, i.e., quantitatively increased or decreased, in control versus experimental cells or conditions. The degree to which expression differs need only be large enough to be detectable via standard characterization techniques.

“Detectable” as used herein, refers to a protein or RNA expression pattern which is detectable via standard techniques, such as, for example, Representational Difference Analysis (RDA). RDA of cDNA is a powerful subtractive hybridization technique that enriches differences between two mRNA populations, thus detecting specific differences in gene expression between control and experimental cells or conditions. Other such standard characterization techniques by which expression differences may be visualized include, but are not limited to microarrays, differential display, reverse transcriptase-(RT-) PCR and/or Northern analyses, which are well known to those of skill in the art.

In order to identify differentially expressed genes, RNA, either total or mRNA, may be isolated from cell populations. RNA samples are obtained from experimental cells and from corresponding control cells. Any RNA isolation technique which does not select against the isolation of mRNA may be utilized for the purification of such RNA samples. See, for example, Sambrook et al. (1989) Molecular Cloning, A Laboratory Manual, Cold Spring Laboratory Harbor Press; Ausubel et al. (1988) Current Protocols in Molecular Biology, John Wiley, both of which are incorporated herein by reference in their entirety. Additionally, large numbers of tissue samples may readily be processed using techniques well known to those of skill in the art, such as, for example, the single-step RNA isolation process disclosed in U.S. Pat. No. 4,843,155 which is incorporated herein by reference in its entirety.

Transcripts within the collected RNA samples which represent RNA produced by differentially expressed genes may be identified by utilizing a variety of methods which are well known to those of skill in the art (see U.S. Pat. No. 6,054,558). For example, differential screening (Tedder et al. (1988) Proc. Natl. Acad. Sci. USA 85, 208-212), subtractive hybridization (Hedrick et al. (1984) Nature 308, 149-153; Lee et al. (1984) Proc. Natl. Acad. Sci. USA 88, 2825-2829), differential display (U.S. Pat. No. 5,262,311) and gene microarray (Lockhart et al. (1996) Nature Biotech. 14, 1675-1680; Schena et al. (1995) Science 270, 467-470). Also for example, and preferably, Representational Difference Analysis (RDA) may be used to identify nucleic acid sequences derived from genes that are differentially expressed This methodology is described by Hubank & Schatz (1994) Nucleic Acids Research 22, 5640-5648; Hubank & Schatz (1999) Methods Enzymol. 303, 325-349) and these references are incorporated herein by reference in their entirety.

Differential Screening

Differential screening involves the duplicate screening of a cDNA library in which one copy of the library is screened with a total cell cDNA probe corresponding to the mRNA population of one cell population while a duplicate copy of the cDNA library is screened with a total cDNA probe corresponding to the mRNA population of a second cell population. For example, one cDNA probe may correspond to a total cell cDNA probe of control cells, while the second cDNA probe may correspond to a total cell cDNA probe of experimental cells. Those clones which hybridize to one probe but not to the other potentially represent clones derived from genes differentially expressed in the control cell or condition versus the experimental cell or condition.

Subtractive Hybridization

Subtractive hybridization techniques generally involve the isolation of mRNA taken from two different sources, e.g., control and experimental cells or conditions, the hybridization of the mRNA or single-stranded cDNA reverse-transcribed from the isolated mRNA, and the removal of all hybridized, and therefore double-stranded, sequences. The remaining non-hybridized, single-stranded cDNA, potentially represent clones derived from genes that are differentially expressed in the two mRNA sources. Such single-stranded cDNA are then used as the starting material for the construction of a library comprising clones derived from differentially expressed genes.

Representational Difference Analysis (RDA)

RDA is a process of subtraction coupled to PCR amplification of cDNA. This technique relies on the generation, by restriction enzyme digestion and PCR amplification, of simplified versions of the mRNA pools under investigation known as “representations”. A control pool (driver) and a test pool (tester) of cDNA are digested with the same restriction enzyme to generate representative fragments likely to contain at least one amplifiable restriction fragment (target) per mRNA species. If a target exists in the tester but not the driver representation, a kinetic enrichment will be achieved by subtractive hybridization of the tester in the presence of excess driver. Sequences with homologues in the driver are rendered unamplifiable, while the target hybridizes only to itself, and retains the ability to be amplified by PCR. Successive iterations of the subtraction/PCR process produce ethidium visible bands on an agarose gel corresponding to enriched target.

The differential display technique describes a procedure, utilizing the well known polymerase chain reaction (the experimental embodiment set forth in U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

Once potentially differentially expressed gene sequences have been identified via bulk techniques such as, for example, those described above, the differential expression of such putatively differentially expressed genes may be corroborated via, for example, such well known techniques as Northern analysis and/or RT-PCR. Upon corroboration, the differentially expressed genes may be further characterized.

Also, amplified sequences of differentially expressed genes may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library. Also, once nucleotide sequence information from an amplified fragment is obtained, the remainder of the gene may be obtained using, for example, RT-PCR.

In one embodiment of such a procedure for the identification and cloning of full length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA/DNA hybrid may then be “tailed” with guanines using a standard terminal transferase reaction, the hybrid may be digested with RNAase H, and second strand synthesis may then be primed with a poly-C primer. Using the two primers, the 5′ portion of the gene is amplified using PCR. Sequences obtained may then be isolated and recombined with previously isolated sequences to generate a full-length cDNA of the differentially expressed genes of the invention.

Microarrays

An “array” or “microarray” refers to a grid system which has each position or probe cell occupied by a defined nucleic acid fragment. The arrays themselves are sometimes referred to as “chips” and “biochips” and “DNA chips” and “gene chips”. High-density DNA microarrays often have thousands of probe cells in a variety of grid styles.

Once the array is fabricated, a batch is added and some form of chemistry occurs between the batch and the array to give some recognition pattern which particular to that array and batch. Autoradiography of radiolabeled batches is a traditional detection strategy, but other options are available, including electronic signal transduction.

Recent advances in cDNA microarray technology enable massive parallel mining of information on gene expression. This process has been used to study cell cycles, biochemical pathways, genome-wide expression in yeast, cell growth, cellular differentiation, cellular responses to a single chemical compound, and genetic diseases, including the onset and progression of the diseases (Schena et al. (1998) Tibtech. 16, 301-302).

The term “marker” refers to any biological-based measurement or observation that is characteristic of a particular biosystem which is being exposed to a particular change, such as a change in temperature, exposure to a chemical or the non-expression of a previously-expressed gene. The term “marker” encompasses both qualitative and qualitative measurements and observations of a biosystem. The marker database constitutes a data set which characterizes gene expression patterns in response to some change, wherein the patterns show which genes are turned on, off, up or down in response to specific change, such as in response to the addition of a composition to the cell(s). Thus, “markers” refers to any biologically-based measurement or observation whose up- and down- or temporal regulations, or qualitative or quantitative changes of expression levels in a biosystem are used to characterize differential biological responses of a biosystem to a change in status.

Examples of markers useful in accomplishing the present invention include, but are not limited to, molecular markers, cytogenetic markers, biochemical markers or macromolecular markers. Macromolecular markers include, but are not limited to, enzymes, polypeptides, peptides, sugars, antibodies, DNA, RNA, proteins (both translational proteins and post-translational proteins), nucleic acids, polysaccharides. Any marker that satisfies the definition of “marker” herein is appropriate for conducting the present invention. The term “markers” includes related, alternative terms, such as “biomarker” or “genetic marker” or “gene marker” or “molecular marker”.

A molecular marker comprises one or more microscopic molecules from one or more classes of molecular compounds, such as DNA, RNA, cDNA, nucleic acid fragments, proteins, protein fragments, lipids, fatty acids, carbohydrates, and glycoproteins.

The establishment, generation and use of applicable molecular markers are well known to one skilled in the art. Examples of particularly useful technologies for the characterization of molecular markers include differential display, reverse transcriptase polymerase chain reactions (PCR), large-scale sequencing of expressed sequence tags (ESTs), serial analysis of gene expression (SAGE), Western immunoblot or 2-D, 3-D study of proteins, and microarray technology. One skilled in the art of molecular marker technology is familiar with the methods and uses of such technology (see, e.g. Bernard et al. (1998) Molecular Biotechnology, Principles and Applications of Recombinant DNA, ASM Press; Walker & Rapley (1997) Route Maps in Gene Technology, Blackwell Science; Roe et al. (1996) DNA. Isolation and Sequencing, John Wiley; Watson et al., (1992) Recombinant DNA, Scientific American Books).

DNA, RNA and protein isolation and sequencing methods are well known to those skilled in the art. Examples of such well known techniques can be found in Sambrook et al. (1989) Molecular Cloning: A Laboratory Manual, Cold Spring Harbor Laboratory Press; Saluz & Jost, (1988) A Laboratory Guide to Genomic Sequencing: The Direct Sequencing of Native Uncloned DNA, Birkhauser; Roe et al. (1996) DNA Isolation and Sequencing, John Wiley. Examples of conventional molecular biology techniques include, but are not limited to, in vitro ligation, restriction endonuclease digestion, PCR, cellular transformation, hybridization, electrophoresis, DNA sequencing, cell culture, and the like. Specific kits and tools available commercially for use in the present invention include, but are not limited to, those useful for RNA isolation, PCR cDNA library construction, retroviral expression libraries, vectors, gene expression analyses, protein antibody purification, cytotoxicity assays, protein expression and purification, and high-throughput plasmid purification.

For discussions, methodologies and applications of oligonucleotide arrays, microarrays, DNA chips or biochips, see, for example, U.S. Pat. Nos. 5,445,934; 5,605,662; 5,631,134; 5,736,257; 5,741,644; 5,744,305; 5,795,714; Schena et al. (1996) Proc. Natl. Acad. Sci. USA 93, 10614-10619; DeRisi et al. (1997) Science 278, 680-686; Wodicka et al. (1997) Nat. Biotech. 15, 1359-1367; Pardee (1997) Nat. Biotech. 15, 1343-1344; Schafer et al. (1998) Nat. Biotech. 16, 33-39; DeRisi et al. (1996) Nature Genetics 14, 457-460; Heller et al. (1997) Proc. Natl. Acad. Sci. USA 94, 2150-2155; Marshall et al. (1998) Nat. Biotech. 16, 27-31; Schena et al. (1998) Tibtech 16, 301-306, Ramsay, (1998) Nat. Biotech. 16, 40-44; Chee et al. (1996) Science 274, 610-614; Chen et al. (1998) Genomics 50, 1-12; Outinen et al. (1998) Biochem. J. 332, 213-221; Gelbert et al. (1997) Curr. Opin. Biotechnol. 8, 669-674.

Protein Analysis

Methods of conventional protein analysis can be used to screen for proteins involved in vascular remodeling. Examples of such methods include, but are not limited to, one- or two-dimensional (2-D) polyacrylamide gel electrophoresis followed by immunoblot, autoradiography staining. See, for example, U.S. Pat. Nos. 5,736,362 & RE 35,747; Coligan et al. (2000) Current Protocols in Protein Science, John Wiley.

Proteomics

The field of proteomics is becoming increasingly important as genome sequences are being completed and annotated. Proteomics investigations endeavor to provide a global understanding of gene product synthesis rate, degradation rate, functional competence, post-translational modification, subcellular distribution and physical interactions with other cell components. For reviews, see, for example, Dutt et al. (2000) Curr. Opin. Biotechnol. 11, 176-179; Gevaert et al. (2000) Electrophoresis 21, 1145-1154; Cash, (2000) Electrophoresis 21, 1187-1201.

A combination of high-resolution two-dimensional (2-D) polyacrylamide gel electrophoresis, highly sensitive biological mass spectrometry, and the rapidly growing protein and DNA databases has paved the way for high-throughput proteomics. Recent advances in proteomics include experimental and mathematical proofs of the need to complement microarray analysis with protein analysis, improved sensitivity for mass spectrometric analysis of separated proteins, better informatic tools for gel analysis and protein spot annotation, first steps towards automated experimental procedures, and new technology for quantitation of protein changes.

Various proteomic methods useful in the present invention include, but are not limited to, two-dimensional gel electrophoresis and mass spectrometric sequencing of proteins to allow the comparison of subsets of expressed proteins among a large number of samples (Johnston-Wilson et al. (2000) Mol. Psychiatry 5, 142-149; Nilsson et al. (2000) Anal. Chem. 72, 2148-2153; Matsumoto et al. (2000) Methods Enzymol. 316, 492-511; Celis et al. (2000) EXS 88, 55-67; comparative protein database analysis (Lai et al. (2000) Genome Res. 10, 703-713); protein and peptide sequencing using wafer-based chip sequencers (Wurzel et al. (2000) EXS 88, 145-157) and biosensor chip mass spectrometry (Nelson et al. (2000) Electrophoresis 21, 1155-1163).

High Throughput Assays

The power of high throughput screening is utilized to the search for new compounds or factors which are involved in the process of angiogenesis. For general information on high-throughput screening (see, for example, Devlin (1998) High Throughput Screening, Marcel Dekker; U.S. Pat. No. 5,763,263). High throughput assays utilize one or more different assay techniques.

Immunodiagnostics and Immunoassays

These are a group of techniques used for the measurement of specific biochemical substances, commonly at low concentrations in complex mixtures such as biological fluids, that depend upon the specificity and high affinity shown by suitably prepared and selected antibodies for their complementary antigens. A substance to be measures must, of necessity, be antigenic -either an immunogenic macromolecule or a haptenic small molecule. To each sample a known, limited amount of specific antibody is added and the fraction of the antigen combining with it, often expressed as the bound:free ratio, is estimated, using as indicator a form of the antigen labeled with radioisotope (radioimmunoassay), fluorescent molecule (fluoroimmunoassay), stable free radical (spin immunoassay), enzyme (enzyme immunoassay), or other readily distinguishable label.

Antibodies can be labeled in various ways, including: enzyme-linked immunosorbent assay (ELISA); radioimmunoassay (RIA); fluorescent immunoassay (FIA); chemiluminescent immunoassay (CLIA); and labeling the antibody with colloidal gold particles (immunogold).

Common assay formats include the sandwich assay, competitive or competition assay, latex agglutination assay, homogeneous assay, microtitre plate format and the microparticle-based assay;

Enzyme-Linked Immunosorbent Assay (ELISA)

ELISA is an immunochemical technique that avoids the hazards of radiochemicals and the expense of fluorescence detection systems. Instead, the assay uses enzymes as indicators. ELISA is a form of quantitative immunoassay based on the use of antibodies (or antigens) that are linked to an insoluble carrier surface, which is then used to “capture” the relevant antigen (or antibody) in the test solution. The antigen-antibody complex is then detected by measuring the activity of an appropriate enzyme that had previously been covalently attached to the antigen (or antibody).

For information on ELISA techniques, see, for example, Crowther (1995) ELISA—Theory and Practice, Humana Press; Challacombe & Kemeny (1998) ELISA and Other Solid Phase Immunoassays—Theoretical and Practical Aspects, John Wiley; Kemeny (1991) A Practical Guide to ELISA, Pergamon Press; Ishikawa (1991) Ultrasensitive and Rapid Enzyme Immunoassay, Elsevier.

Colorimetric Assays for Enzymes

Colorimetry is any method of quantitative chemical analysis in which the concentration or amount of a compound is determined by comparing the color produced by the reaction of a reagent with both standard and test amounts of the compound, often using a colorimeter. A calorimeter is a device for measuring color intensity or differences in color intensity, either visually or photoelectrically.

Standard colorimetric assays of beta-galactosidase enzymatic activity are well known to those skilled in the art (see, for example, Norton et al. (1985) Mol. Cell. Biol. 5, 281-290). A colorimetric assay can be performed on whole cell lysates using O-nitrophenyl-beta-D-galactopyranoside (ONPG, Sigma) as the substrate in a standard colorimetric beta-galactosidase assay (Sambrook et al. (1989) Molecular Cloning—A Laboratory Manual, Cold Spring Harbor Laboratory Press). Automated colorimetric assays are also available for the detection of beta-galactosidase activity, as described in U.S. Pat. No. 5,733,720.

Immunofluorescence Assays

Immunofluorescence or immunofluorescence microscopy is a technique in which an antigen or antibody is made fluorescent by conjugation to a fluorescent dye and then allowed to react with the complementary antibody or antigen in a tissue section or smear. The location of the antigen or antibody can then be determined by observing the fluorescence by microscopy under ultraviolet light.

For general information on immunofluorescent techniques, see, for example, Knapp et al. (1978) Immunofluorescence and Related Staining Techniques, Elsevier; Allan (1999) Protein Localization by Fluorescent Microscopy—A Practical Approach (The Practical Approach Series) Oxford University Press; Caul (1993) Immunofluorescence Antigen Detection Techniques in Diagnostic Microbiology, Cambridge University Press. For detailed explanations of immunofluorescent techniques applicable to the present invention, see U.S. Pat. Nos. 5,912,176; 5,869,264; 5,866,319 & 5,861,259.

### EXAMPLES

Overview

Recent studies have shown that cytolytic T lymphocytes are the primary effector cells of acute graft rejection in human transplantation, and microvascular endothelial cells (EC) are the major cellular targets of alloreactive CTL-mediated injury in rejecting human allografts. Cultured HUVEC have been previously used to study the susceptibility of human endothelial cells to CTL and other killer cell populations. Herein we disclose the effect of overexpression of caspase-resistant Bcl-2 in HUVEC on resistance to injury mediated by CTL, and upon the survival and maturation of the synthetic vascular bed that develops upon transplantation of EC cultured in a 3-D collagen/fibronectin matrix.

The caspase-resistant Bcl-2 (or control) retroviral vector was constructed and stably transduced into isolated cultured cells by repetitive infections using supernatants produced by a packaging cell line. As indicators of normal function, cell growth was measured in terms of cell number, and the expression of endothelial cell markers and of the transduced DNA (Bcl-2 or a control DNA) were quantitated by flow cytometry. Bcl-2 protection against cell death was assessed in response to apoptosis inducers, to serum and growth factor withdrawal, and to CTL-mediated killing.

After transduction and maintenance in traditional two-dimensional cell culture, these cells were suspended in a buffered solution containing collagen and fibronectin, which was then allowed to gel. Immature tubules formed within this three-dimensional matrix, and upon contact with an established vascular bed, inosculation occurred and the tubules began to mature. These events and factors involved in vascular remodeling may be investigated by analysis of differential gene expression.

### Example 1

Isolation and Culture of HUVEC Cells

HUVEC were isolated by collagenase treatment of human umbilical veins as previously described (Gimbrone (1976) Prog. Hemostasis Thromb. 3, 1-6) and cultured on 0.2% gelatin-coated plastic in Medium 199 with 20% FCS, 50 μg/ml endothelial cell growth supplement (ECGS) (Collaborative Research/Becton Dickinson), 100 μg/ml heparin (Sigma), 2 mM L-glutamine, 100 U/ml penicillin, and 100 μg/ml streptomycin. All of the EC used in these experiments were at passage levels 1 through 6. Such cultures are homogeneous for EC markers (von Willebrand factor, CD31, inducible E-selectin) and are free of contaminating CD45⁺ leukocytes.

### Example 2

Construction of the Retroviral Vector Expressing Caspase Resistant Bcl-2

The D34A caspase-resistant form of Bcl-2 DNA (SEQ ID NO: 1) in the pSG5 expression vector has been described (Cheng et al. (1997) Science 278, 1966-1968). The 800 bp cDNA insert was isolated by PCR and subcloned into the pCRII vector. DNA sequence of the insert of subclone #10 indicated the following terminal sequences: 5′-AATTCGGATCA CGGTCA CCAATGGCGCACGCT (SEQ ID NO: 3) . . . CTGAGCCACAAGTGA GTCGACCTCGAGGAATTC-3′ (SEQ ID NO: 4). The EcoRI sites (GAATTC and the translation start (ATG) and stop (TGA) codons are underlined. The EcoRI excisable DNA insert was subcloned into the LZRSpBMN-Z retroviral vector. This retroviral vector DNA containing the caspase resistant form of Bcl-2 DNA was directly transfected into the Phoenix-Ampho packaging cell line by lipofection and puromycin-resistant cells were derived which served as the source of retroviral stocks.

To generate a control retroviral vector, Enhanced Green Fluorescent Protein (EGFP) was inserted into the LZRSpBMN-Z retroviral vector.

### Example 3

Stable Transduction of Caspase-Resistant Bcl-2 or Control DNA

Infection of HUVEC was accomplished by four serial infections over two weeks without drug selection (Inaba et al. (1997) J. Surg. Res. 78, 31-36). In brief, standard viral infections in the presence of polybrene (5 μg/ml) were performed for six hours with 1×10⁵ HUVEC at passage one. The normal growth medium was replaced and cells were maintained overnight. The infection was repeated the next day. Cells were carried in culture for a week and then the process of double infection repeated starting with 1×10⁵ cells. Control transductions used the EGFP-encoding retroviral vector, or no retroviral vector. In general, each single retroviral infection produced 30-50% stably transduced cells. By performing two double cycles of infection, early passage HUVEC lines were reproducibly generated of which at least 95% of the cells expressed the expected cDNA.

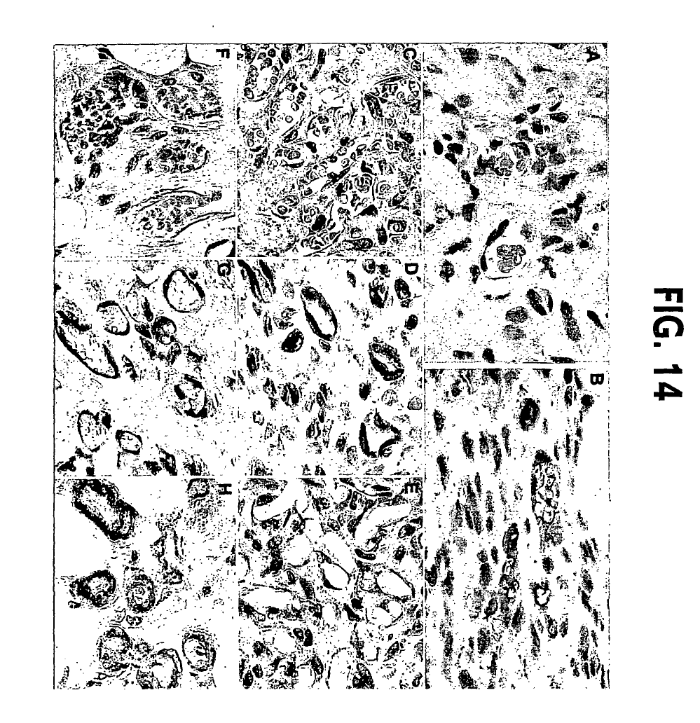

Alternatively, the infection of HUVEC can be accomplished by serial infections over one or more weeks using drug selection (for example, using G418). Drug selection is necessary to achieve the very high levels of transduction of PAEC as used in the procedure summarized in FIG. 14. For examples of G418-based drug selection of transduced cells, see Rio et al. (1999) Gene Ther. 6, 1734-1741; Scott-Burden et al. (1996) Circulation 94, 235-238; Townsend et al. (1996) Am. Surg. 62, 619-624.

### Example 4

Analysis of Bcl-2 Effects on Normal Cell Growth and Gene Expression

Growth Analysis

2×10⁴ HUVEC, untransduced or stably transduced with either control or Bcl-2 cDNA, were plated in replicate wells of a 24-well plate. Starting at the day of seeding (day 0) through day eight, six wells were quantitatively harvested at each time point and aliquots were counted with a hemocytometer. The mean and SEM of cell number/well was calculated. The remaining cells from the replicate wells were pooled and stained with propidium iodide and used to assess the cell cycle status by flow cytometry (Al-Ramadi et al. (1998) Proc. Natl. Acad. Sci. USA 95, 12498-12501).

Under standard culture conditions, serially passaged HUVEC require both ECGS and 10-20% serum for growth and survival. Cells plated at subconfluent densities in the presence of serum and growth factor divide about every 30 hours until confluence is reached. At this point, cell division is reduced but does not completely cease. Cell numbers in confluent cultures remain roughly constant because cells detach and undergo anoikis at about the same rate as cells divide. Confluent cultures remain as a strict monolayer of flattened cells without significant overlapping. Both Bcl-2 and EGFP-transduced HUVECs displayed this characteristic growth behavior of normal HUVECs and were indistinguishable from each other. No further increase in cell number was observed at later times (data not shown). Both types of transduced HUVEC also displayed indistinguishable distributions throughout the cell cycle as assessed by propidium iodide analysis. Thus, Bcl-2 did not appear to confer any growth advantage, nor did Bcl-2 transduced cells show any evidence of transformed cell behavior (e.g., piling up or focus formation at confluence) under optimal culture conditions.

Flow Cytometric Analysis of Protein Expression

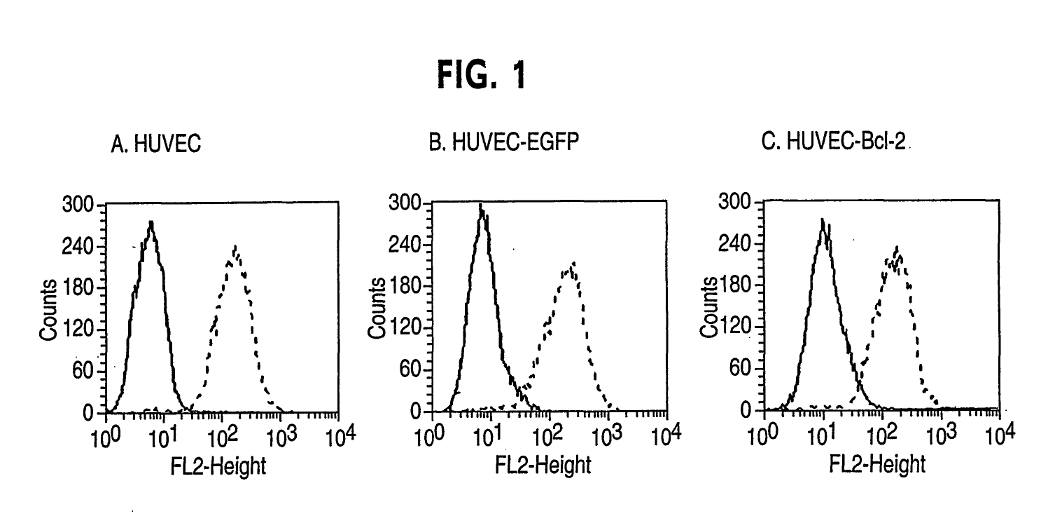

Expression of endothelial cell markers on non-permeabilized HUVEC was measured by indirect immunofluorescence flow cytometric analysis as described previously using a FACScan (Becton Dickenson) flow cytometer and CELLQUEST software (Kluger et al. (1997) J. Immunol. 158, 887-901). The primary antibodies used were the W6/32 mAb for MHC class I, the H4/18 mAb for E-selectin, and non-binding K16/16 mAb was used as a negative control. Cells that were transduced with Bcl-2 or with the control cDNA showed similar FACS profiles for MHC class I expression; essentially unchanged compared to cultures not subjected to retroviruses (see FIG. 1).

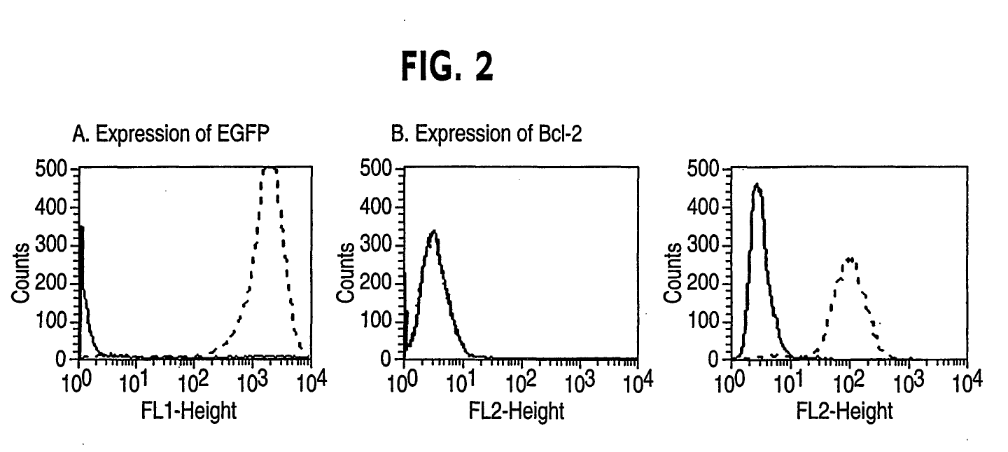

Expression of Bcl-2 in fixed and permeabilized HUVEC was measured by indirect immunofluorescence flow cytometric analysis. HUVEC were fixed with 4% paraformaldehyde for ten minutes at room temperature and washed twice. Cells were permeabilized with PBS with 0.1% saponin (Sigma) and 1% BSA for ten minutes at room temperature and then incubated with anti-human Bcl-2 mAb (clone 124, DAKO) in PBS with 0.1% saponin for 60 minutes at room temperature. A nonbinding IgG mAb (Jackson Immunoresearch) was used as an isotype control. Cells were then washed twice with PBS with 0.1% saponin and incubated with R-Phycoerythin (PE)-conjugated Donkey anti-mouse IgG (1/100, Jackson Immunoresearch Lab) in PBS with 0.1% saponin for 30 minutes at room temperature. After incubation; cells were washed twice, suspended in 0.5 ml of PBS, and analyzed using a FACScan flow cytometer and CELLQUEST software (see FIG. 2B). Expression of EGFP in HUVEC was directly measured by fluorescence flow cytometric analysis (see FIG. 2A).

The levels of EGFP and of Bcl-2 expression in representative cultures are shown in FIG. 2. EGFP fluorescence (FIG. 2A) was approximately 3.5×10³ greater than background, and Bcl-2 staining (FIGS. 2B-b) was approximately 1.5×10³ greater than background (FIGS. 2B-a).

### Example 5

Analysis of Bcl-2 Effects on Apoptosis

Quantitation of Transduced-Cell Resistance to Apoptosis-Inducing Agents or Serum Withdrawal

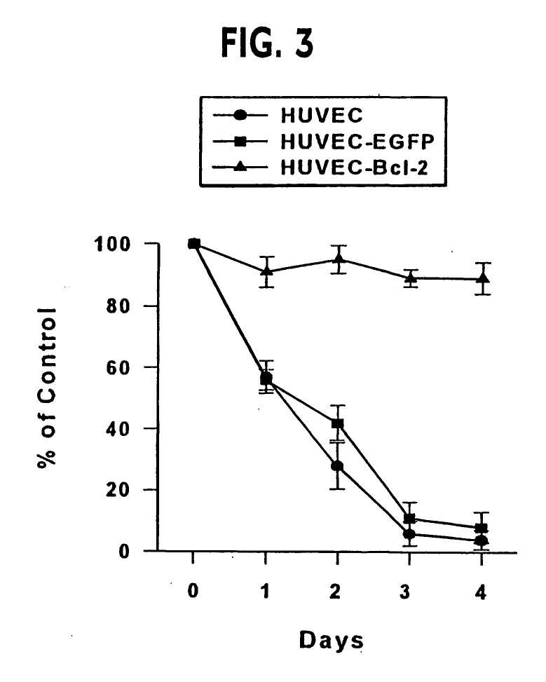

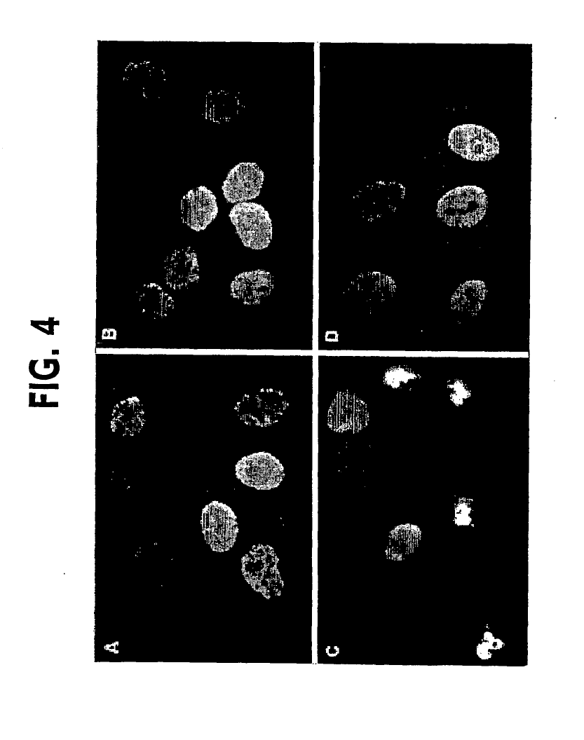

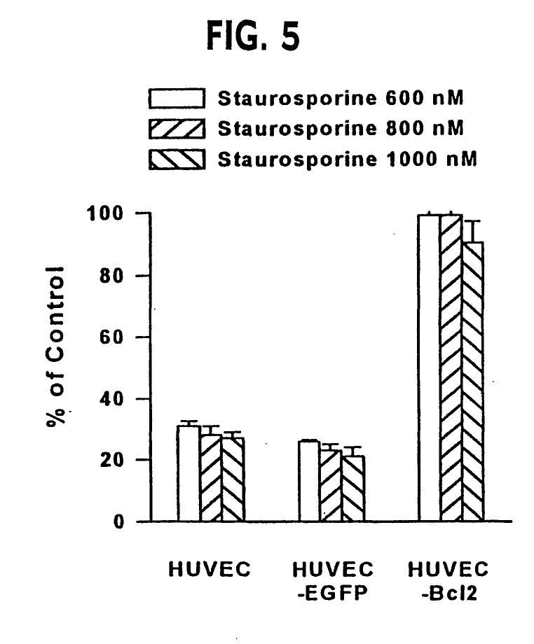

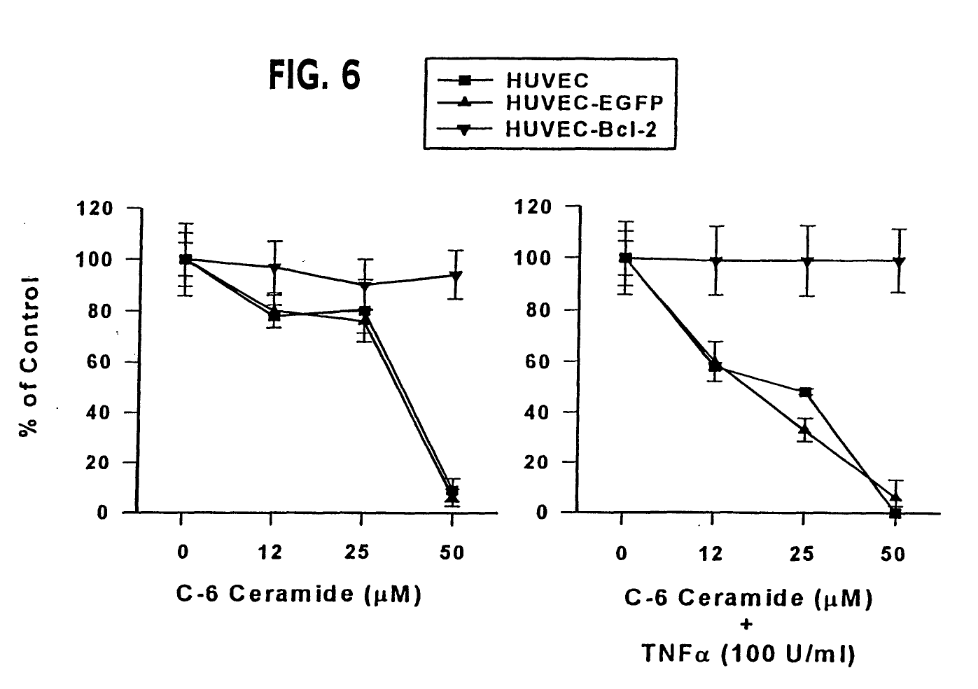

HUVEC were plated at 2×10⁴ cells/200 μl Medium 199 with 20% FCS and ECGS in 96-well flat-bottom plates coated with 0.20% gelatin. After overnight incubation, HUVEC were incubated with the apoptosis inducers staurosporin (Calbiochem) (see FIG. 5), C6-ceramide (Matrya Inc.) (see FIG. 6) and/or TNF-α (R&D Systems) at the indicated concentrations and incubated overnight. Where indicated, ceramide effects were potentiated by co-addition of TNF (Ridge et al. (1998) Nature 393, 474-476) (see FIG. 6). In experiments to study serum and growth factor withdrawal, Medium 199 lacking serum and ECGS was added (see FIGS. 3 and 4).

In both types of experiments, resistant HUVEC, which remained attached to the wells, were quantitated by DNA measurement. Specifically, at the indicated times, the wells were rinsed twice in PBS to remove dead cells, and the adherent resistant cells were incubated in 70% ethanol containing 100 μg/ml Hoechst 33258 (Molecular Probes) for thirty minutes at room temperature. Each well was then rinsed twice with PBS, and the retained fluorescence was quantified in a fluorescence plate reader (PerSeptive Biosystems).

Qualitative Assessment of Cell Death—DAPI Staining

To characterize the pattern of cell death, nuclear morphology was assessed by DAPI staining and fluorescence microscopy. HUVEC were plated at 3.5×10⁵ cells/3 ml Medium 199 with 20% FCS and ECGS in six-well plates coated with 0.2% gelatin and incubated overnight. HUVEC were washed with Medium 199 and incubated with Medium 199 in the presence or absence of serum and ECGS. After overnight incubation, HUVEC were then harvested and spun onto gelatin-coated glass slides by Cytospin (Cytospin 2, Shandon) for three minutes at 800 rpm. Cells were fixed with 100% methanol for three minutes at room temperature. After washing the slides in PBS, cells were incubated with 0.1 μg/ml 4′,6-diamidino-2-phenylindole, dihydrochloride (DAPI) (Molecular Probes) in PBS for five minutes. After incubation, the slides were washed in PBS for ten minutes, air dried, and embedded in mounting medium. Cells were examined and photographed with a fluorescence microscope (Microphot FXA, Nikon) (see FIG. 4).

Withdrawal of serum and growth factor from HUVEC cultures caused growth arrest and an increase in the number of cells undergoing apoptosis for at least 4 days after treatment was initiated. Under such conditions, HUVEC overexpressing Bcl-2 showed no change in cell number whereas EGFP-transduced and uninfected cells detached from the plate (see FIG. 3). Furthermore, compared to EGFP, transduced Bcl-2 protein protected HUVEC from apoptotic cell death detected by nuclear condensation and fragmentation in DAPI-stained cells after 24 hours (see FIG. 4). Despite the absence of cell death, Bcl-2 transductants showed no signs of proliferation in the absence of serum and growth factor and were unchanged in appearance for the duration of the experiment.

The effects of a variety of treatments that actively induce apoptosis were also evaluated (Slowik et al (1997) Lab Invest. 77, 257-267; Madge et al. (1999) J. Biol. Chem. 274, 13643-13649). Twenty-four hour treatment with three different concentrations of staurosporine had no effect on survival of the D34A Bcl-2 transductants while the EGFP-expressing control cells were highly sensitive and detached from the culture dish (see FIG. 5). In addition, treatment with ceramide with or without TNF showed that the D34A Bcl-2 transductants were completely resistant to these agents as well whereas the EGFP transductants again were sensitive (see FIG. 6). Nuclear morphology of DAPI-stained cells again confirmed that the control cells died by a process of apoptosis that was prevented by D34A Bcl-2 protein (data not shown). Cumulatively, these data show that D34A Bcl-2 conferred resistance to apoptosis mediated by neglect or in response to injury without influencing cell growth.

Quantitation of CTL-Mediated Killing

To assess the effect of Bcl-2 expression on CTL-mediated injury, cytolysis of Bcl-2 or EGFP-transduced HUVEC was examined with either the total PBMC effector population or with purified CD4 and CD8 T cells purified from the pool.

Generation and Purification of BLCL.

B lymphoblastoid cells lines (BLCL) were generated from cord blood mononuclear cells (PBMC) harvested from the same individual as the HUVEC as previously described. Briefly, cord blood PBMC were isolated by density gradient centrifugation using lymphocyte separation medium (LSM, Organon Teknika). BLCL were generated by transformation of PBMC with Epstein Barr virus and cultured in RPMI 1640 in 10% FCS with 2 mM L-glutamine, 100 U/ml penicillin, and 100 μg/ml streptomycin for four to six weeks.

Generation and Purification of CTL

1×10⁶ allogeneic, γ-irradiated (100 Gy) BLCL were co-cultured with 10×10⁶ PBMC isolated as described previously (Paavonen et al. (1992) Transplant Proc. 24, 342-343. in six-well plates in RPMI 1640 with 10% human AB serum (Irvine Scientific), 10 U/ml recombinant human IL-2 (Life Technologies), 2 mM L-glutamine, 100 U/ml penicillin and 100 μg/ml streptomycin. Co-cultures were fed with fresh medium containing 10 U/ml IL-2 after three days and restimulated weekly with allogeneic, γ-irradiated BLCL in medium containing 10 U/ml IL-2 at a ratio of stimulator/responder=1/10. After two to three weeks, the resultant cells were either tested directly for CTL activity or used as a source for positive selection of CD4 and CD8 T cells for CTL assay.

CD4 and CD8 lymphocytes were positively selected from the bulk CTL lines using anti-CD4 or anti-CD8 Ab-coated magnetic beads (CD4 and CD8 Positive Isolation Kits, Dynal) according to the manufacturer's instruction. Briefly, effector cells from bulk culture were harvested, suspended at 1×10⁷ cells/ml in PBS with 2% FCS, and incubated with 5×10⁷ per ml Dynabeads conjugated with anti-CD4 or CD8 mAb for twenty minutes at 4° C. Bead-bound cells were isolated using a magnet, washed four to five times in PBS with 2% FCS, and resuspended in RPMI 1640 with 1% FCS. Detachabead solution was added to the cell suspension, which was then incubated for 45-60 minutes at room temperature. The detached CD4 or CD8 T cells were recovered and the purity of these T cell subsets was >95% as assessed by direct immunofluorescence flow cytometric analysis.

Assay of CTL-Mediated Killing

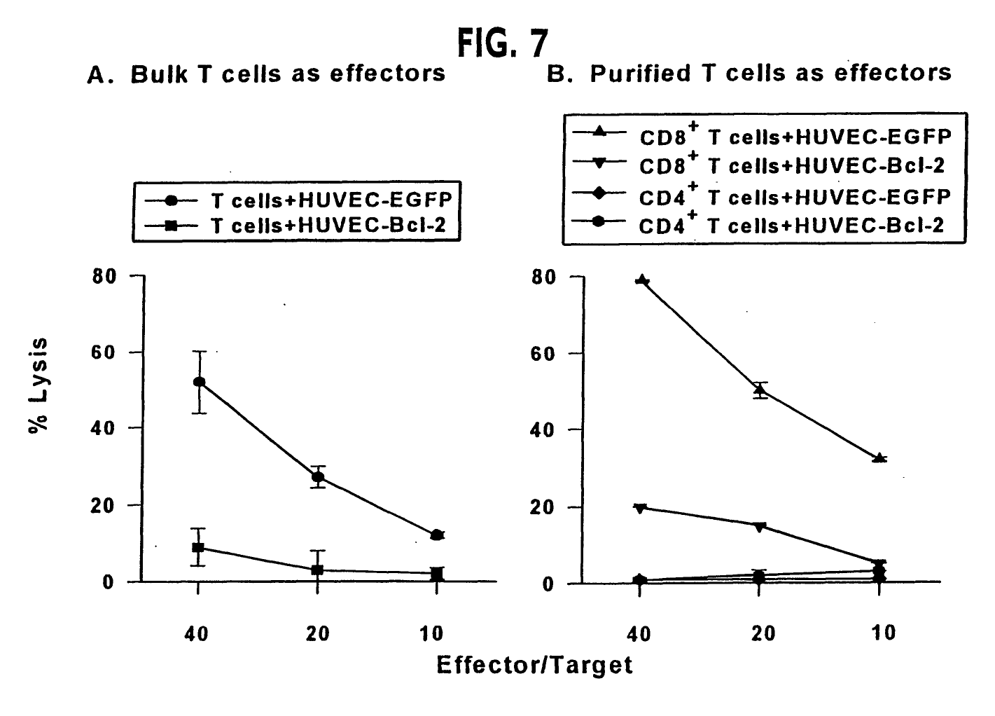

Target cell lysis was assessed by a calcein fluorescence release assay as previously described (Biedermann et al. (1998) J. Immunol. 161, 4679-4687). The transduced HUVEC targets were plated at 2×10⁴ cells/200 μl in 96-well flat-bottom plates coated with 0.2% gelatin and incubated overnight. Cells were then incubated with 50 μM Calcein-AM (Molecular Probes) in M199 with 5 mM HEPES for thirty minutes at 37° C. and washed twice with Medium 199 with 5% FSC, 5 mM HEPES, 2 mM L-glutamine, 100 U/ml penicillin and 100 μg/ml streptomycin. Effector cells from bulk culture were washed once, and added at various E/T ratios to calcein-loaded HUVEC targets at 200 μl/well in triplicates and incubated at 37° C. (see FIG. 7).

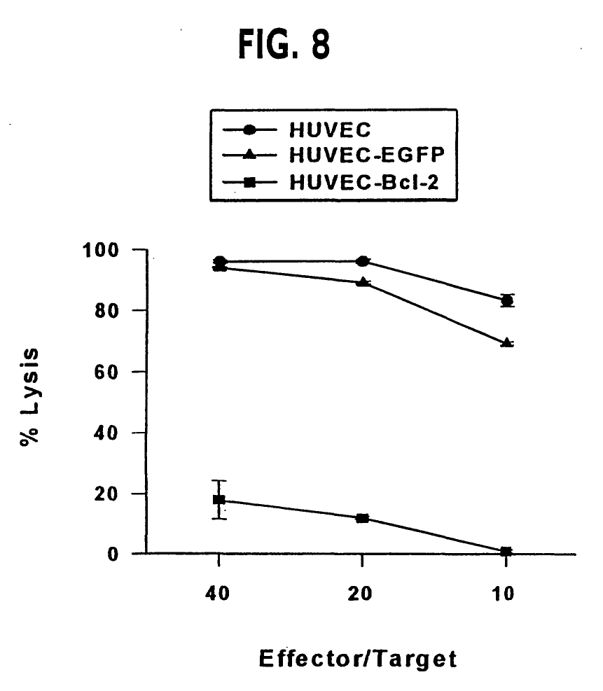

In the redirected CTL assay, the cytolytic activity was measured in the presence of 5 μg/ml of PHA (phytohaemagluttinin) using transduced HUVEC targets derived from donors different from those used to generate the BLCL stimulators. After a four hour incubation, retained calcein was measured using a fluorescence multi-well plate reader (Cytofluor2, Perseptive Biosystems) at an excitation wavelength 485 nm and emission wavelength 530 nm. Percent specific killing was calculated as: 100−(retained sample−maximal retained)÷(spontaneous retained−maximal retained)×100% (see FIG. 8).

As shown in FIG. 7, the total population produced about 50% lysis at a 40:1 E:T ratio on the EGFP transduced cells while only about 10% lysis was observed with the Bcl-2 transduced cells. HUVEC lysis by CTL was predominantly an apoptotic process as assessed by DAPI staining (data not shown). When the effector cell populations were purified, all of the lytic activity was associated with the CD8 T cells and maximum lysis increased to 80% for EGFP and 20% for Bcl-2 transduced cells.

In addition, we examined the effects on CTL activity in the presence of the activating lectin PHA. This agent results in lysis that is independent of allorecognition. In FIG. 8, the control HUVEC and EGFP-HUVEC showed almost complete lysis while the Bcl-2 transductants were very effectively protected from lysis in the redirected lysis assay. Caspase-resistant Bcl-2 overexpression therefore does render HUVEC resistant to killing by CTL.

The results show that retroviral vector mediated overexpression of Bcl-2 in HUVEC has no effect on cell growth or on other pathophysiological EC responses (e.g. TNF-mediated activation) but does protect HUVEC from various inducers of apoptotic cell death. Most significantly, overexpression of caspase-resistant D34A Bcl-2 is able to strongly reduce the extent of killing by alloreactive CTL.

In summary, we have demonstrated that retroviral vector mediated overexpression of Bcl-2 in HUVEC confers protection against apoptotic cell death and CTL mediated killing without altering the cell growth and activation responses. Gene therapy with Bcl-2 may represent a potentially attractive approach for prevention of immune rejection in transplantation. Graft EC are accessible to the organ perfusion solution ex vivo and new methods for effective transduction (e.g., lentivirus or AAV) of resting cells are now available for clinical use.

### Example 6

Formation of Vascular Constructs in Vitro

HUVEC rapidly undergo apoptosis when suspended in type I collagen gels (Ilan et al. (1998) J. Cell. Sci. 111, 3621-3631 and unpublished observations). Therefore, our initial studies were aimed at delaying apoptosis by suspending early passage HUVEC in a mixed collagen-fibronectin gel, combining the structural properties of type I collagen fibers with the cell adhesive and survival enhancing properties of fibronectin (Fukai et al. (1998) Exp. Cell Res. 242, 92-99; Maciag et al. (1982) J. Cell Biol. 94, 511-520).

Untransduced HUVEC cells were harvested from traditional two-dimensional culture and suspended in a solution of rat tail type 1 collagen (1.5 mg/ml), and human plasma fibronectin (90 μg/ml, both from Collaborative Research), in 25 mM HEPES and 1.5 mg/ml NaHCO₃ buffered Medium 199 (Sigma) at 4° C. pH was adjusted to 7.5 using 0.1 M HCl. pH may alternatively be neutralized before the addition of endothelial cells to the cold collagen solution. The HUVEC suspension was pipeted into rat tail type 1 collagen coated C-6 transwells (Collaborative) and warmed to 37 C. for ten minutes to allow polymerization of the collagen. Warmed Medium 199 supplemented with 20% fetal bovine serum, and 50 μg/ml EC growth factor, 200 U/ml penicillin, 200 μg/ml streptomycin, 2 mM L-glutamine, and 100 μg/ml heparin was added to the transwells, to cover the solidified gels. In some experiments the gels were maintained in culture for as long as seven days, without further growth supplementation.

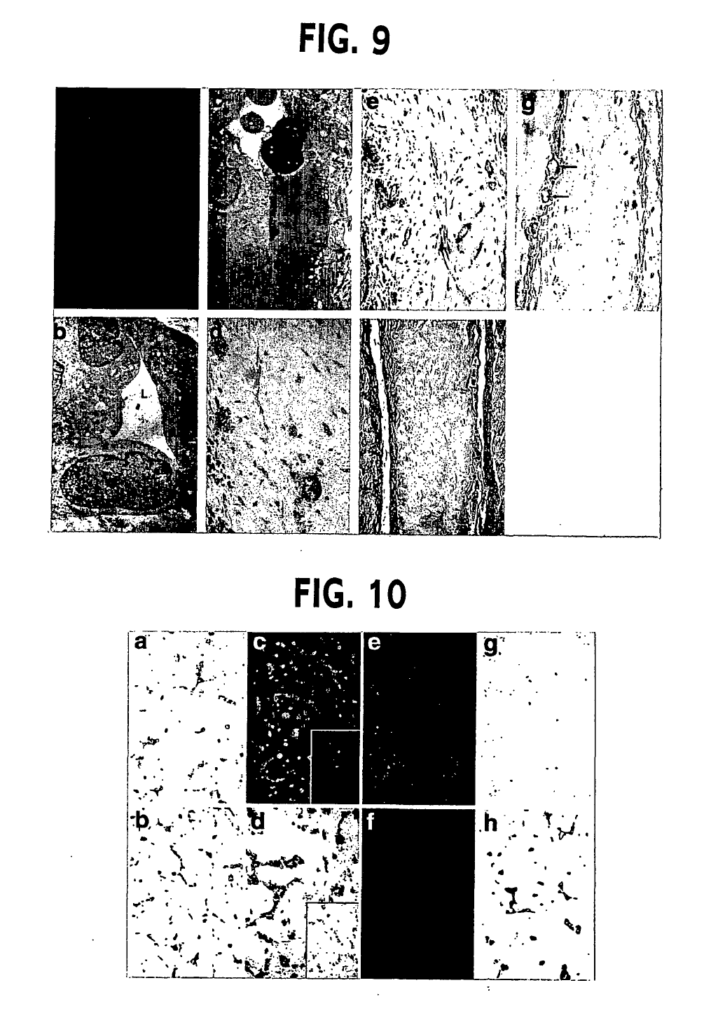

By eighteen hours in this gel culture, isolated HUVEC spontaneously reorganized into multicellular cords (FIG. 9A), and by 24 hours these cords appeared to be in the early stages of developing lumena free of collagen fibers (FIG. 9B). However, by 24 hours significant numbers of EC showed morphologic evidence of apoptosis (FIG. 9C), and by 48 hours, essentially all of the HUVEC had died. Early subcultures of HUVEC appeared to form better tubes than cells passaged more than two times (not shown).

### Example 7

Implantation of Vascular Constructs and Analysis of Inosculation and Maturation Implantation

For implantation into animals (Schnecher et al. (2000) Proc. Natl. Acad. Sci. USA 97, 9191-9196), gels were harvested and trisected approximately twenty hours after formation. Each resulting 1×1×0.2 cm gel segment was implanted into a bluntly dissected subcutaneous pouch in the anterior abdominal wall of a five to eight week old SCID/beige mouse (Taconic). The wound was closed with skin staples. At the indicated time, typically 31 or 60 days, the constructs were harvested, and analyzed by conventional histology, immunohistochemistry, and/or electron microscopy.

Immunocytochemistry

Double antibody staining was performed on 4 μm thick frozen sections with anti-smooth muscle α-actin mAb (1A4, Sigma) and biotinylated Ulex europaeus agglutinin I (UEA-I, Vector Laboratories) using standard detection techniques (Schechner et al. (1999) Lab. Invest. 79, 601-607). Single antibody staining was performed on 3 μm thick formalin fixed, paraffin embedded sections using, anti-Bcl-2 (DAKO) or anti-smooth muscle α-actin mAb or UEA-1 lectin, followed by a light hematoxylin stain. Isotype-matched non-binding antibodies were utilized in all antibody staining experiments to control for non-specific reactivity.

Electron Microscopy

Tissue was fixed in Karnovsky's fixative, and processed as described (Slowik et al. (1996) Circ. Res. 79, 736-47). For in vivo experiments cardiac perfusion with the fixative was performed on anesthetized animals. Sections were viewed on a Zeiss EM 910 electron microscope at 80 kV.

Data Collection and Statistical Analysis

The number of vessels per area of gel was calculated by dividing the number of endothelial-lined spaces that contained erythrocytes within the entire gel, in hematoxylin and eosin stained formalin-fixed tissue sections, by the cross sectional area of the gel. One or two observers blinded to treatment protocol counted the vascular profiles. The cross sectional area of the gels was obtained from video microscopy images using NIH image software. All specimens were stained with UEA-1 to insure that greater than 99% of the vascular profiles were lined by human endothelium. Statistical analyses of significance were performed using a paired t-test.

We subcutaneously implanted twenty hour HUVEC-derived synthetic “vascular beds” into eleven SCID-beige mice. Constructs harvested 31 days after implantation contained thin walled tubes filled with erythrocytes, consistent with perfusion by the mouse circulation (FIG. 9D). These vascular profiles were present in ten of the eleven constructs at a mean density of 124.1±2.8 per 10⁵ μm². As assessed by UEA-1 lectin staining, the majority of tubes were wholly composed of human EC (FIG. 9E). In contrast, anti-mouse CD31 antibodies reacted with fewer than 1% of the vascular profiles (data not shown) within the constructs, confirming that the vessel-like structures were not formed by mouse neovascularization of the gel. Vascular profiles failed to develop if the HUVEC did not form cords prior to implantation, and first or second passage level HUVEC were consistently superior to later passage HUVEC in forming perfused vascular profiles in vivo (data not shown). In mock constructs, which contained no HUVEC, there were no detectable vascular profiles except at the very edges of these empty collagen gels 31 days after implantation (FIG. 9F). We conclude that HUVEC-derived cords formed in vitro, survive, evolve into tubes, and inosculate with mice microvessels at the gel boundary, acquiring perfusion.

Since apoptosis limited cord formation in vitro, we used the above transduction culture and implantation techniques to evaluate whether further inhibition of apoptosis would improve the performance of the synthetic microvessel constructs. When cultured in a collagen/fibronectin gel for 24 hours, both the EGFP- and Bcl-2-transduced cells readily form into cords similar in appearance to non-transduced HUVEC (FIGS. 10A and B). EGFP-transduced HUVEC display green autofluorescence 24 hours after incorporation into the constructs, indicating expression of EGFP (FIG. 10C), whereas, immunohistochemistry confirmed the expression of the Bcl-2 transgene in Bcl-2 transduced cells (FIG. 10D). By 36 hours in gel culture, few intact EGFP-transduced HUVEC tubes remained (FIG. 10E), while those formed from Bcl-2-transduced HUVEC continued to elongate (FIG. 10F). By day seven in culture, no viable EGFP-transduced HUVEC were detectable (FIG. 10G), while the Bcl-2-transduced EC maintained capillary like structures (n=3, FIG. 10H). Thus, Bcl-2 overexpression effectively increased the persistence of HUVEC-derived cords in collagen/fibronectin gel culture.

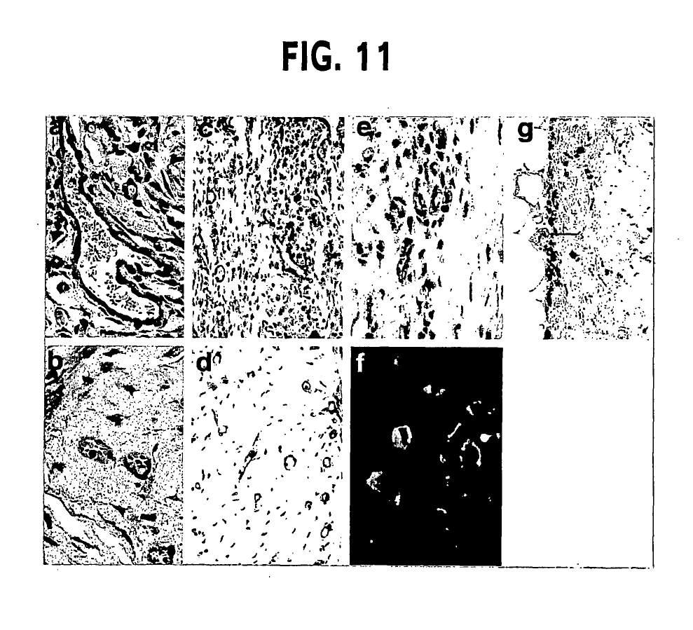

Ten mice were implanted with 18 to 24 hour vascular constructs containing EGFP-transduced and 11 with Bcl-2-transduced HUVEC. By 31 days after implantation into mice, transduced HUVEC constructs developed perfused human endothelial-lined vascular profiles (FIGS. 11A-D), and the tubular structures maintained expression of the transduced gene products in vivo (FIGS. 11D & E). However, there were several striking morphologic differences between Bcl-2- and the EGFP-transduced vascular constructs. Overexpression of Bcl-2 significantly increased the density of vascular structures to an average of 431.5±19.9, compared to 81.5±22.6 vascular profiles per 10⁴ μm² in the EGFP group (p=1.6×10⁻⁷). The endothelial-lined structures formed from Bcl-2 overexpressing cells showed a much greater variation in size and shape, with visible branching, than those formed from EGFP-transduced cells (FIGS. 11A-B).

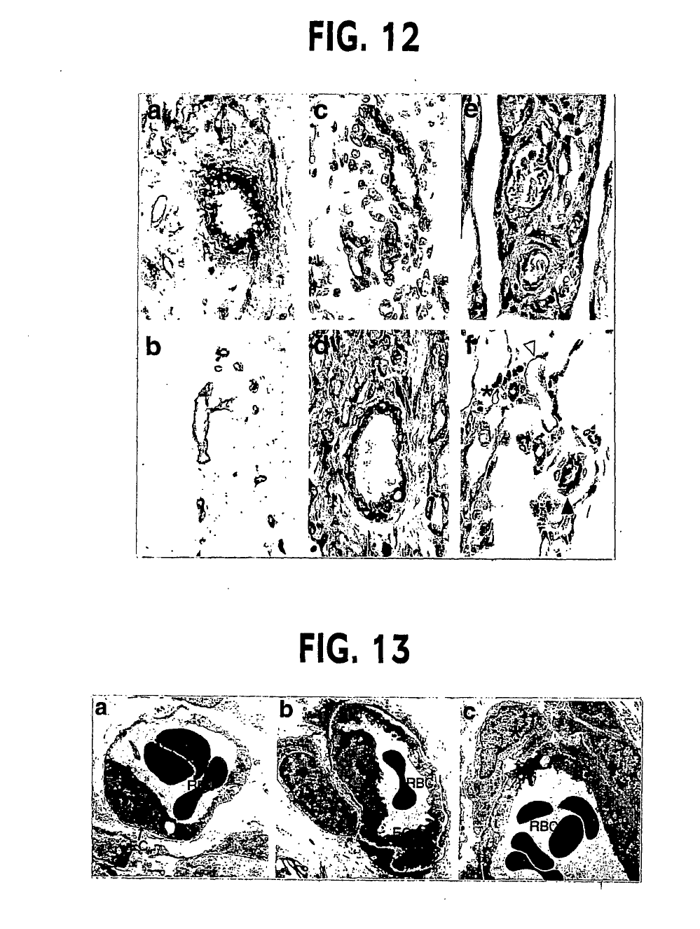

In addition, many of the vascular structures lined by Bcl 2-transduced HUVEC appeared to have two or more cell layers. The inner layer was composed of Bcl-2-, UEA-1-expressing human endothelial cells, but the outer investing layers were UEA-1 and Bcl-2 negative, and smooth muscle α-actin positive (FIGS. 12A-C). No myosin thick filaments were detected in these investing cells by electron microscopy, consistent with their identity as pericytes or incompletely differentiated vascular smooth muscle cells (FIGS. 13A-C). These investing cells appear to have been recruited from the surrounding mouse tissue because the density of extravascular cells was greatest at the periphery of the constructs, especially evident in a limited number of specimens harvested only thirteen days after implantation. Similar structures were not observed in any of the constructs transduced with EGFP or in those of previous experiments using HUVEC that had not been transduced.

Seven additional constructs containing Bcl-2-transduced cells were harvested sixty days after implantation. In six of these specimens the HUVEC-lined vascular structures were organized into complex vascular beds with elements that closely resembled arterioles, venules and capillaries (FIGS. 12D-E). Using the same immunohistochemical analyses applied at 30 days, we found that these vessels were comprised of HUVEC surrounded by supporting cells of mouse origin. Thus, Bcl-2 transduction promotes both HUVEC survival, and enhanced vascular remodeling, resulting in the formation of mature vascular beds.

Similar experiments were done with porcine aortic endothelial cells (PAEC) transduced with a caspase-resistant (D34A) form of Bcl-2 or with EGFP. Two mice were implanted with EGFP-PAEC for one month, two others with Bcl-2-PAEC, and three mice were implanted with Bcl-2-PAEC for two months. The H+E staining shows vessels formed in all gels recovered. At one month there are a large number of vessels in the Bcl-2-PAEC and fewer in the GFP-PAEC vessels. At two months the Bcl-2-PAEC vessels were larger. The EC stained appropriately for either EGFP-PAEC (see FIG. 14B) or Bcl-2 (FIGS. 14D and G). At one month there is recruitment of smooth muscle-like cells in Bcl-2-PAEC vessels (FIG. 14G) but it is significantly greater at two months (FIG. 14H).

### Example 8

Implantation of Vascular Constructs and Analysis of Inosculation and Maturation into Bcl-2 Transduced Mice

The procedures of Example 7 are repeated except that one or more cells of the SCID/beige mice have been transduced with Bcl-2 prior the implantation of the gels into the mice. Preferably, the transduced cells of the mice are those that are in direct contact with or in close proximity to the implanted gel.

In another variation of this experiment, the Bcl-2 used to transduce the mouse cells prior to implantation is a caspase-resistant Bcl-2.

In still another variation of this experiment, the mice are transduce for Bcl-2, preferably caspase-resistant Bcl-2, prior to the implantation of the gel. In this version, every cell of the mice should theoretically contain the Bcl-2 coding sequence prior to the implantation of the gel.

### Example 9

Identifying Genes Expressed Selectively During Vascularization or Vascular Remodeling

Techniques that screen for differential expression may be used to identify genes and gene products involved in the recruitment of smooth muscle cells and/or in other aspects of vascularization or vascular maturation. Differentially expressed genes and proteins are detected by comparing the pattern of expression in cells undergoing vascular remodeling (experimental or test cells) and cells that are not undergoing remodeling (control cells). Herein, the experimental cells may be endothelial cells expressing a DNA that codes for a factor that promotes vascular remodeling, for example the caspase-resistant Bcl-2 mutant known as D34A. The control cells may be the equivalent endothelial cell population except that they are not transduced at all, or are transduced with control DNA that does not promote vascular remodeling, such as DNA encoding EGFP.

Alternatively, experimental cells may be genetically indistinguishable from control cells, for example of the same cell type and similarly transduced or untransduced. In this case the treatment of the two cell populations would distinguish them as experimental and control cells. A non-limiting example would be endothelial cells that are transduced with wild-type Bcl-2 and dispersed in a 3-D gel matrix that does (experimental) or does not (control) contain a component that promotes or suppresses vascular remodeling. Such a component could be, but is not limited to collagen of any type, fibronectin, other ECM proteins, or factors.

Experimental cells may also be compared to control cells that are unrelated cells (e.g., fibroblasts) that are also subject to the experimental treatment, in order to screen out generic effects on gene expression that might not be related to vascular remodeling. Such generic effects might be manifest by changes in gene expression that are common to the experimental cells and the unrelated cells that are subject to the same experimental treatment.

Before the RNA populations are harvested for comparison, the control and experimental cell populations may be maintained in any of a variety of culture conditions. For example they may be grown in conventional (two-dimensional) cell culture, or in vitro in 3-D-culture, or in vivo in 3-D cell culture (for example, following transplantation of a 3-D cell culture construct into an animal).

When the technique used to screen for differential gene expression is Representational Difference Analysis (RDA), then the experimental cells would be used to generate the test pool of mRNA (tester), and the control cells used to generate the control pool of mRNA (driver) as set forth above in the Detailed Description of the Preferred Embodiments.

### Example 10

Microarray Printing

Gene clones which comprise genes from various tissues can be obtained from sources such as the IMAGE Consortium libraries through Research Genetics. Most clones have been partially sequenced and are available as expressed sequence tags form the dbEST database of GenBank. Clones comprising pBluescript plasmids can be separately cultured and amplified using commercially available primers prior to application on nylon membranes (Chen et al. (1998) Genomics 51, 313-324). Approximately 10 ng of each amplified target can be applied on a positively charged nylon membrane using a computer controlled arraying system. Roughly 85,000 spots can be placed on a piece of nylon membrane measuring 35 by 55 mm using a 24-pin arraying tool.

RNA samples can be obtained from experimental cells and from corresponding control cells as set forth above in the Detailed Description of the Preferred Embodiments. The experimental and control cells is set forth above in Example 8. For example, the experimental cells can be endothelial cells expressing a DNA that codes for a factor that promotes vascular remodeling, for example the A34 mutant of Bcl-2. The control cells may be the equivalent endothelial cell population except that they are not transduced at all, or were transduced with control DNA, such as DNA encoding EGFP.

In one specific example, mRNA is isolated from the experimental cells and the control cells both before and either during or after exposure to the 3-D constructs of the present invention. In another specific example, the mRNA is isolated from the experimental and control cells before and either during or after they are exposed to an agent of interest.

One microgram of each isolated mRNA sample is labeled with biotin and/or digoxigenin using random primed reverse transcription. The labeled samples are treated with alkali and the resulting labeled nucleic acids are precipitated prior to use in hybridization. Membrane hybridization and washing can be carried out using the labeled probes as disclosed in Chen et al. (1998) Genomics 51, 313-324. To detect the spots on the membrane in dual color mode (i.e., both biotin and digoxigenin), β-galactosidase-conjugated streptavidin (Strept-Gal) and alkaline phosphatase-conjugated digoxigenin antibody (anti-Dig-AP) can be employed. After color development, image digitization using a imaging means is employed (e.g., a flatbed scanner or digital camera).

Quantitative measurements are determined by computer analysis which uses a program that measures the integrated density of the primary color components of each spot, performs regression analysis of the integrated density data and locates statistical outliers as differentially expressed genes.

In this manner, we are able to correlate specific gene expression with the exposure of a cell to no, low (L) or high (H) amounts of an herbal composition. Many of the genes identified in this way code for proteins important in known metabolic or biochemical pathways. Many of these proteins have direct and indirect effects on certain physiological, morphological and psychological parameters. Thus, this method permits the association of a particular genetic fingerprint of an herbal composition with its array biological effects. Such associations can be used to profile or characterize an herbal composition for the purposes of Quality Control and Quality Assurance and evaluating pharmacological or toxicological properties. The role of primary and secondary herbs in an herbal formula can also be assessed by this approach.

### Example 11

Construction of Synthetic Skin

Synthetic vascular beds will be used to increase the extent of perfusion and thereby improve survival of synthetic skins. Endothelial cells transduced with caspase-resistant Bcl-2 may be suspended in a dermal equivalent that is a collagen-based matrix containing fibronectin or other matrix components that enhance the survival of incorporated cells, reduce immunogenicity, or enhance the structural integrity of the engineered skin. Examples of such additional matrix components include vitronectin, fibrin, laminin, and additional collagen subtypes types as well as proteoglycans such as dermatan sulfate.

The dermal equivalent may include cells other than endothelial cells, which may or may not be genetically modified. These cells will be added to improve the overall survival and engraftment of the constructs, as well as to add functionality. These cells may include, but are not limited to fibroblasts and smooth muscle cells. An alternative strategy is to use acellular human or porcine dermis as the matrix rather than a synthetic matrix. If this strategy is employed, endothelial and possibly other cell types will be allowed to grow into, rather than be initially suspended in, the matrix.

Whatever matrix strategy is used, cultured keratinocytes will be placed on the surface of the constructs, and subjected to conditions that promote differentiation into a stratified epidermis.

### Example 12

Direct Injection of Bcl-2 Transduced Endothelial Cells

In an alternative method, the solution comprising collagen, fibronectin and the Bcl-2 transduced endothelial cells can be directly injected into animals, including humans. Thus, the constructs discussed herein can be directly injected into an animal of choice so as to form synthetic vascular beds in an effort to promote vascularization/revascularization in situ.

In another modification of this direct injection procedure, the Bcl-2 used to transduce the endothelial cells is a caspase-resistant Bcl-2, as discussed elsewhere herein (see, for example, the procedure of Example 2 above).

Basically, the injected transduced cells are suspended in the fibronectin/collagen matrix prepared as discussed elsewhere herein (see, for example, the matrix discussed in Example 6, above). As long as the solution is kept cold, it will remain in a liquid state. When the solution reaches the target tissue it will warm to body temperature and subsequently solidify.

This methodology will be utilized to provide a higher degree of vascularization to a variety of tissues and organs in which increased perfusion may be beneficial.

### Example 13

Direct Injection of Bcl-2 Transduced Endothelial Cells into Bcl-2 Transduced Animals

The procedures of Example 12 are repeated except that one or more cells of the recipient animal have been transduced with Bcl-2 prior to the direct injection of the solution into the animal. Preferably, the transduced cells of the animal are those that are in direct contact with or in close proximity to the injected solution.

In another variation of this experiment, the recipient animal cells have been transduced with a caspase-resistant Bcl-2 prior to direct injection of the solution of the present invention.

In still another variation of this experiment, the recipient animals are transgenic for Bcl-2, preferably caspase-resistant Bcl-2, prior to the direct injection of the solution of the present invention. In this version, every cell of the animal should theoretically contain the Bcl-2 coding sequence prior to the direct injection of the solution of the present invention.

### Example 14

Revascularization of Acellular Dermis with Human Eendothelial Cells

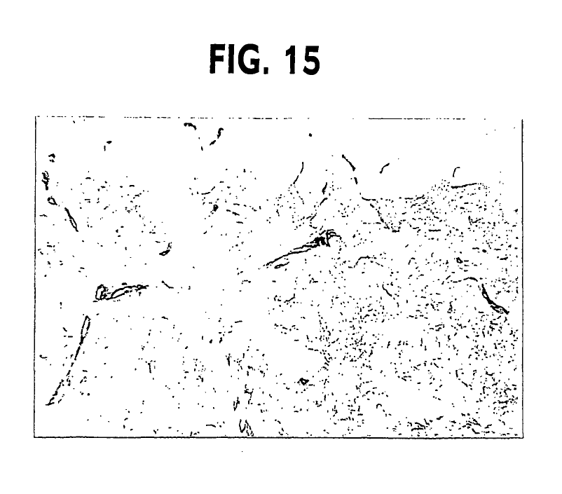

Human umbilical vein endothelial cells (HUVEC) are used to seed acellular dermis, which when implanted into immunodeficient mice, form new vessels that become perfused by the mouse blood. Acellular dermis is derived from split thickness cadaveric human skin grafts, which have been incubated in sterile saline for one month in order to allow the death of all the native cellular constituents. One cm² pieces of the acellulardermis are placed into collagen coated transwells such that the former epidermal surface is face down. HUVEC are suspended in medium 199 supplemented with 20% fetal calf serum, penicillin, streptomycin, glutamine, and endothelial cell growth supplement at a concentration of 2.5×10⁶ cells per ml. Cloning disks, with a capacity of 300 μl, are placed on top of the acellular dermis, and are filled with the HUVEC suspension such that a density of approximately 8×10⁵ cells is introduced per one cm² piece of dermis. The cloning disks are removed after 24 hours, and the seeded dermal pieces are left in culture for an additional one to three days. During the first two to four days in culture, prior to implantation in mice, the HUVEC migrate into the acellular dermis, appearing to line the existing vascular channels (FIG. 15).

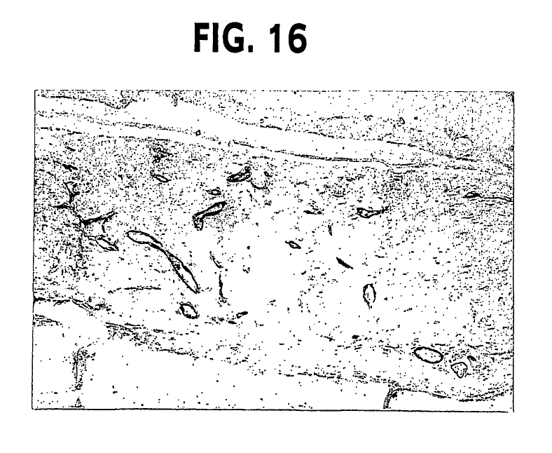

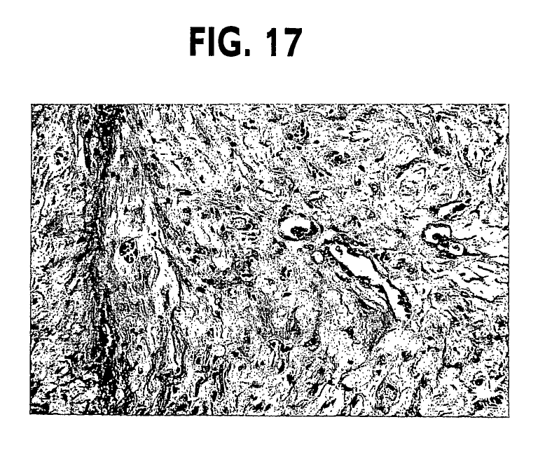

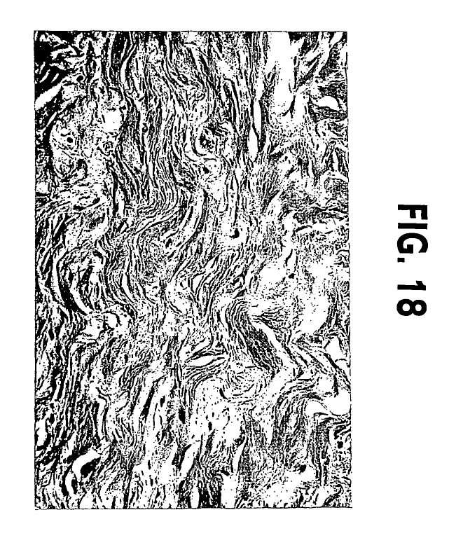

Seeded grafts are placed subcutaneously into scid/beige mice after one to three days of in vitro culture. Within one month after implantation into scid/beige mice, the acellular dermis seeded with HUVEC contain perfused vascular structures lined by human endothelial cells (FIGS. 16 and 17). Human endothelial cells survive within these vascular structures for at least two months following subcutaneous implantation into scid/beige mice. If the acellular dermis is not seeded with endothelial cells, the grafts do not become vascularized (FIG. 18).

### Example 15

In Vivo Revascularization of Acellular Dermis with Modified Human Endothelial Cells

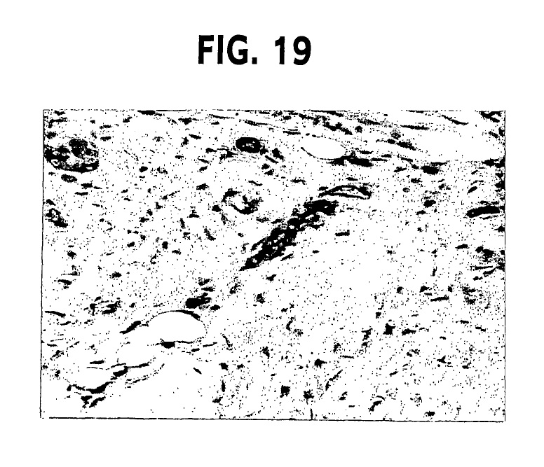

A method for vascularizing acellular dermis with genetically modified human endothelial cells was developed. Retroviral transduction was utilized to express either a caspase resistant form of the survival gene Bcl-2, or a control transgene, EGFP HUVEC. These cells were then seeded on acellular human dermis. Within three days, it appeared that existing vascular channels became repopulated with the genetically modified HUVEC. Dermis seeded with either Bcl-2 or EGFP transduced HUVEC (n=9) were implanted subcutaneously into SCID/beige mice. To assess the effect of the presence of fibroblasts on revascularization, five of the constructs in each group were also seeded with human dermal fibroblasts. One month after implantation the grafts were harvested, revealing that the majority contained perfused vascular profiles. UEA-1 staining wag utilized to confirm that observed vascular structures were lined by human endothelium. Immunostaining demonstrated continued transgene expression in vivo (FIG. 19). Blinded scoring of vascular density on a 0-5 scale revealed that the implants seeded with Bcl-2 transduced HUVEC had a higher mean score (3.0±0.7) compared to the EGFP controls (1.6±0.6). No beneficial effect of additionally seeding with fibroblasts was observed.

### Example 16

Overexpression of Bcl-2 in an in Vivo Model of Low Grade Angiosarcoma

An in vivo model of low-grade angiosarcoma was developed and utilized to evaluate the effects Bcl-2 in combination with a transforming gene. The murine endothelial cell line MS-1 was transformed with SV40 and suspended in a collagen/fibronectin matrix. This resulted in the in vitro formation of vascular cords. When implanted subcutaneously into SCID-beige mice, tumors composed of dense networks of perfused vascular structures, containing EC with hyperchromatic nuclei, and intravascular endothelial hyperplasia, were observed. Retroviral transduction was utilized to overexpress a caspase resistant form of Bcl-2, or the control transgene EGFP, in the EC incorporated into collagen/fibronectin matrix prior to implantation in mice (n=9). Thirty days after implantation, five mice in each group were harvested. The morphology of the resultant vascular networks in both groups was similar to those which had not been transduced. Although the mean Bcl-2 transduced tumor volume (71.9±19.7 mm³) was greater than those transduced with EGFP (34.0±26.5 m³) at thirty days, sixty days after implantation, there was no significant volume difference between the Bcl-2-transduced (60.2±23.3 mm³) and the EGFP-transduced (77.5±19.65 mm³) tumors (p=0.78, n=4). No gross or microscopic metastases were detected in either group. Local invasion was minimal, Although slightly greater extension of the atypical vascular structures into the abdominal wall musculature was observed in the Bcl-2 transduced tumors.

### Example 17

Perfusi n of Acellular Dermis Pieces Pre-Seeded with Human Keratinocytes

One significant application of the technology of vascularizing acellular dermis is the perfusion of functional synthetic skin grafts. Living skin equivalents currently do not truly engraft onto the recipient bed, but aid in wound healing by acting as a biologic dressing. This lack of engraftment is probably due to inadequate perfusion in the post implantation period, since grafts are not revascularized by the recipient for at least ten days. This is particularly problematic in patients with impaired capacity for angiogenesis such as those with diabetes or chronic leg ulcers. The methodology for seeding acellular dermis with human keratinocytes, inducing differentiation into a stratified epithelium, and implanting these into mice is well established. To date, there has been no success in vascularizing these skin equivalents. We have adapted the methodology for vascularizing acellular dermis to perfusing acellular dermis pieces that have been pre-seeded with a human keratinocytes.

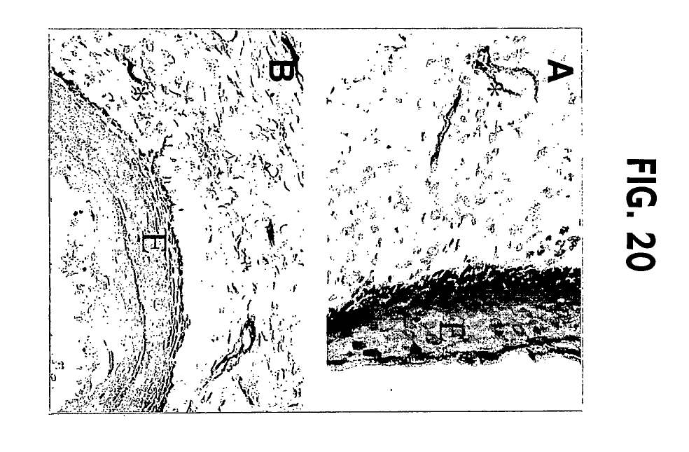

Human keratinocytes derived from neonatal foreskins are seeded on the epidermal side of a 1×1 cm acellular dermis, and incubated for forty-eight hours in complete KBM-2 media (Clonetics) supplemented with penicillin and streptomycin. To induce stratification and differentiation of the epidermis, the media is then changed to 60% KBM-2, 30% DMEM, and 10% F-12 in media, supplemented with 10% fetal calf serum, cholera toxin, EGF, hydrocortisone, penicillin and streptomycin. After an additional three to seven days in culture, the acellular dermis constructs are seeded with HUVEC (untransduced and Bcl-2 transduced) as previously described. They are incubated for an additional two days in complete Media 199, or complete Media 199 supplemented with cholera toxin, EGF and hydrocortisone, and implanted subcutaneously into SCID/beige mice. After 30 days in the mice the grafts were observed to have vessels, lined by human endothelial cells, that were perfused by mouse blood, and contained a stratified epidermis formed from the human keratinocytes (FIG. 20). This example proves that the described methodology can produce a functional vascularized human skin equivalent.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

We claim:

1. A synthetic composition comprising collagen, fibronectin, and at least one cell.

2. The synthetic composition of claim 1 wherein the cell is an endothelial cell which comprises a nucleic acid encoding a cleavage-resistant Bcl-2 polypeptide.

3. The synthetic composition of claim 2 wherein the nucleic acid encodes a caspase-resistant Bcl-2 polypeptide designated D34A.

4. The endothelial cell of claim 2 wherein the endothelial cell is derived from a human umbilical vein, a human dermal microvascular bed, adipose tissue or from porcine tissues.

5. The composition of claim 1 wherein the composition is a solution capable of gelation, a semi-solid solution capable of gelation, or a gel.

6. The composition of claim 1 wherein the collagen is Type I collagen.

7. The composition of claim 1 wherein the fibronectin is human plasma fibronectin.

8. A construct prepared by a method comprising the following steps:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide; and
(c) polymerizing the collagen within the solution of step (b) to form a three-dimensional gel.

9. A construct prepared by a method comprising the following steps:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide;
(c) adjusting the solution of step (b) to between about pH 7.0 and about pH 8.0; and
(d) warming the solution of step (c) to between about 25° C. and about 40° C. to form a three-dimensional gel.

10. A method of animal implantation comprising implanting into an animal the construct produced by the method of claim 9.

11. The method of claim 10 wherein the animal is an immunodeficient animal.

12. The method of claim 11 wherein the immunodeficient animal is a SCID or SCID/beige mouse.

13. A method for forming endothelial cells into tubes within a matrix which comprises the steps of:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide;
(c) polymerizing the collagen within the solution of step (b) to form a matrix; and
(d) allowing the endothelial cells to form tubes within the matrix.

14. A method of producing endothelial cell tubules in vivo which comprises the steps of:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide;
(c) warming the suspension of step (b) so that the collagen gels to produce a three-dimensional gel;
(d) polymerizing the collagen within the solution of step (b) to form a three-dimensional gel; and
(e) implanting the three-dimensional gel produced in step (d) into an animal.

15. The method of claim 14 wherein the animal is an immunodeficient animal.

16. The method of claim 15 wherein the immunodeficient animal is a SCID or SCID/beige mouse.

17. The method of claim 14 wherein the endothelial cell tubules have one or more characteristics of mature microvessels.

18. The method of claim 14 wherein the endothelial cells are derived from the animal into which the three-dimensional gel is subsequently implanted.

19. The method of claim 14 wherein the endothelial cell tubules are perfused by blood.

20. The endothelial cell tubules produced by the method of claim 14.

21. The endothelial cell tubules of claim 20 wherein the endothelial cell tubules are perfused by blood.

22. A method of promoting vascularization in a tissue or an organ, comprising the steps of:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide;
(c) polymerizing the collagen within the solution of step (b) to form a three-dimensional gel;
(d) allowing the endothelial cells in the three-dimensional gel of step (c) to form tubules; and
(e) contacting the tubule-containing three-dimensional gel of step (d) with a tissue.

23. The method of claim 22 wherein the tissue or organ is used for engraftment.

24. The tissue of claim 22 wherein the tissue is synthetic skin.

25. A method for identifying genes or gene products involved in the process of vascularization comprising:
(a) obtaining a first construct comprising endothelial cells wherein the construct promotes vascularization;
(b) obtaining a second construct comprising endothelial cells wherein the construct does not promote vascularization; and
(c) identifying gene products detectable in one but not both constructs.

26. A method for identifying gene or gene products involved in the process of vascular remodeling comprising:
(a) obtaining a first construct comprising endothelial cells wherein the construct promotes vascular remodeling;
(b) obtaining a second construct comprising endothelial cells wherein the construct does not promote vascular remodeling; and
(c) identifying gene products detectable in one but not both constructs.

27. A method of identifying an agent which modulates vascularization or vascular remodeling comprising the steps of:
(a) exposing a construct to an agent, wherein the construct comprises endothelial cells and promotes vascularization or vascular remodeling; and
(b) determining whether the agent modulates vascularization or vascular remodeling.

28. The method of claim 25 or 26 or 27 wherein the constructs are in vivo or in vitro.

29. A method of promoting vascularization in an animal, comprising the steps of:
(a) preparing a solution comprising collagen and fibronectin;
(b) suspending endothelial cells in the solution of step (a) wherein the suspended endothelial cells comprise a nucleic acid encoding a caspase-resistant Bcl-2 polypeptide; and
(c) directly injecting the solution of step (b) into an animal.
